(19) United States
(12) Patent Application Publication
(10) Pub. No.: US 2023/0130375 A1
(43) Pub. Date: Apr. 27, 2023

(54) TOPOLOGIES OF SYNTHETIC GENE CIRCUIT FOR OPTIMAL FOLD CHANGE ACTIVATION

(71) Applicant: TECHNION RESEARCH & DEVELOPMENT FOUNDATION LIMITED, Haifa (IL)

(72) Inventors: Ramez DANIEL, Haifa (IL); Phyana LITOVCO, Haifa (IL); Roee SAMUEL, Haifa (IL); Doaa NAFFAA, Haifa (IL)

(21) Appl. No.: 17/974,805

(22) Filed: Oct. 27, 2022

Related U.S. Application Data
(60) Provisional application No. 63/272,389, filed on Oct. 27, 2021.

(51) Int. Cl. A61K 48/00; C12N 15/63

(52) U.S. Cl. CPC A61K 48/005; C12N 15/63

(57) ABSTRACT

The present invention is directed to systems including an expression vector or a plurality thereof, including methods of using same, such as for controlling the expression level of a gene of interest (GOI) or reducing high basal expression level of a GOI.

### CROSS-REFERENCE TO RELATED APPLICATIONS

This application claims the benefit of priority of U.S. Provisional Patent Application No. 63/272,389, titled “TOPOLOGIES OF SYNTHETIC GENE CIRCUIT FOR OPTIMAL FOLD CHANGE ACTIVATION”, filed 27 Oct. 2021, the contents of which are incorporated herein by reference in their entirety.

### FIELD OF INVENTION

The present invention is in the field of molecular biology and genetic engineering.

### BACKGROUND

The main challenge of genetic regulatory networks implemented in various synthetic gene circuits in living cells for purpose of sensing, computing, and actuating in fields of diagnostics, health monitoring, environmental monitoring, bioremediation, and metabolic engineering, is the ability to control cellular behavior in modular, robust, and accurate ways.

Untight control of transcriptional regulatory networks applied on promoters, which are the basic transcriptional regulatory component in synthetic circuits, often exhibit high basal level expression or leaky expression of target promoter or part under test (P_{PUT}). In this process, RNAPs bind non-specifically to the target promoter, even in the absence of its inducer molecule, which result in the promoter activity called leakiness. This leakiness also affects the fold change activation (FCA) of the target promoter. FCA is defined as the ratio between the ‘ON’ state, when an activated promoter has maximum activity, and the ‘OFF’ state (background level or basal level), when the promoter has minimum activity. Promoters with high FCA exhibit distinct ON and OFF states in contrast to circuits with low FCA, which are sensitive to environmental changes, have a narrow noise margin and thus, demonstrate poor performance. Tight control of genes involved in metabolic pathways is required to yield high amounts of the desired products in metabolic engineering.

In addition, high basal level expression of enzymes can lead to toxicity to the cells if the expressed protein is toxic. Another aspect of biosensors having high basal level expression is that they often show low sensitivity for detecting target molecule at very low concentrations. To address these problems various biomolecular tools were implemented such as, integrating constitutive expression of a repressor of the target promoter, use a lower copy number plasmid, incorporation of mutations at ribosome binding sites (RBS), addition of degradation tags to decrease protein's half-time, random mutagenesis, hybrid promoter engineering, using a different host cell, antisense transcription, etc.

There is still a great need for a synthetic gene circuit for optimal fold change.

### SUMMARY

According to a first aspect, there is provided a system comprising at least 2 expression vectors, the system comprising: (a) a first expression vector comprising a first promoter sequence operably linked to a first nucleic acid sequence encoding a gene of interest (GOI) and a second nucleic acid sequence encoding a polynucleotide or a protein product thereof, characterized by being capable of inhibiting or reducing expression of a regulatory RNA polynucleotide, an activity thereof, or both, wherein the first promoter is responsive to an input signal; and (b) a second expression vector comprising a second promoter sequence operably linked to a nucleic acid sequence encoding the regulatory RNA polynucleotide, wherein the second promoter is constitutively active, and wherein the regulatory RNA polynucleotide inhibits or reduces expression levels of the GOI.

According to another aspect, there is provided a system comprising at least 3 expression vectors, the system comprising: (a) a first expression vector comprising a first promoter sequence operably linked to a first nucleic acid sequence encoding a GOI and a second nucleic acid sequence encoding a polynucleotide or a protein product thereof, characterized by being capable of inhibiting or reducing expression of a first regulatory RNA polynucleotide when complexed with a second regulatory RNA polynucleotide, an activity thereof, or both, wherein the first promoter is responsive to an input signal; (b) a second expression vector comprising a second promoter sequence operably linked to a nucleic acid sequence encoding the first regulatory RNA polynucleotide, wherein the second promoter is constitutively active, and wherein the first regulatory RNA polynucleotide inhibits or reduces expression levels of the GOI; and (c) a third expression vector comprising the second promoter sequence operably linked to a nucleic acid sequence encoding the second regulatory RNA polynucleotide.

According to another aspect, there is provided a system comprising an expression vector comprising: (a) a first promoter sequence operably linked to a first nucleic acid sequence encoding a GOI, a second nucleic acid sequence encoding a protein translation regulatory element, and a third nucleic acid sequence encoding a protein characterized by being capable of binding to a second promoter sequence; and (b) a second promoter; wherein the first promoter is responsive to an input signal, wherein the protein characterized by being capable of binding to the second promoter sequence represses transcription from the second promoter, wherein the second promoter is constitutively active, and wherein the second promoter transcribes in a direction opposite to the first promoter sequence.

According to another aspect, there is provided a system comprising: (a) a first expression vector comprising a first promoter sequence operably linked to a first nucleic acid sequence encoding a GOI, a second nucleic acid sequence encoding a transcription factor capable of activating transcription of a second promoter, and a third nucleic acid sequence encoding a first regulatory RNA polynucleotide capable of inhibiting or reducing expression levels of the GOI, the transcription factor, or both, wherein the third nucleic acid sequence is located between the first nucleic acid sequence and the second nucleic acid sequence; and (b) a second expression vector comprising the second promoter sequence operably linked to a nucleic acid sequence encoding a second regulatory RNA polynucleotide, wherein the second promoter is constitutively active, and wherein the second regulatory RNA polynucleotide inhibits or reduces expression of the first regulatory RNA polynucleotide of the first expression vector, an activity thereof, or both.

According to another aspect, there is provided a cell comprising the system disclosed herein.

According to another aspect, there is provided a method for controlling expression level of a GOI operably linked to an inducible promoter in a cell, wherein the promoter is responsive to an input signal, the method comprising contacting the cell of the invention with an effective amount of an agent triggering or providing the input signal, thereby controlling expression level of the GOI operably linked to an inducible promoter in a cell.

According to another aspect, there is provided a method for reducing high basal expression level of a GOI operably linked to an inducible promoter in the absence of an input signal while preserving high expression level of the GOI in the presence of the input signal, wherein the promoter is responsive to the input signal, and wherein the GOI expression is controlled by at least two negative feedback loops, the method comprising contacting a cell comprising the GOI operably linked to the inducible promoter, and having expression being controlled by at least two negative feedback loops, with an effective amount of an agent triggering or providing the input signal, thereby reducing high basal expression level of the GOI operably linked to an inducible promoter in the absence of an input signal while preserving high expression level of the GOI in the presence of the input signal.

According to another aspect, there is provided a method for reducing high basal expression level of a GOI operably linked to an inducible promoter in the absence of an input signal while preserving high expression level of the GOI in the presence of the input signal, wherein the promoter is responsive to the input signal, and wherein the GOI expression is controlled by an indirect coherent feedforward loop, the method comprising contacting a cell comprising the GOI operably linked to the inducible promoter, and having expression being controlled by an indirect coherent feedforward loop, with an effective amount of an agent triggering or providing the input signal, thereby reducing high basal expression level of the GOI operably linked to an inducible promoter in the absence of an input signal while preserving high expression level of the GOI in the presence of the input signal.

In some embodiments, the first expression vector further comprises a third nucleic acid sequence encoding a protein translation regulatory element.

In some embodiments, the third nucleic acid sequence is located between the first nucleic acid sequence and the second nucleic acid sequence.

In some embodiments, the system further comprises a third expression vector comprising the first promoter sequence operably linked to a nucleic acid sequence encoding the polynucleotide or the protein product thereof, characterized by being capable of inhibiting or reducing expression of the regulatory RNA polynucleotide of the second expression vector, an activity thereof, or both.

In some embodiments, the expression vector is devoid of the nucleic acid sequence encoding the polynucleotide or the protein product thereof, characterized by being capable of inhibiting or reducing expression of the regulatory RNA polynucleotide of the second expression vector, an activity thereof, or both.

In some embodiments, the first expression vector further comprises a third nucleic acid sequence encoding a protein translation regulatory element.

In some embodiments, the third nucleic acid sequence is located between the first nucleic acid sequence and the second nucleic acid sequence.

In some embodiments, the system further comprises a fourth expression vector comprising the first promoter sequence operably linked to the nucleic acid sequence encoding the polynucleotide or a protein product thereof, characterized by being capable of inhibiting or reducing expression of the first regulatory RNA polynucleotide when complexed with the second regulatory RNA polynucleotide, an activity thereof, or both.

In some embodiments, the first expression vector is devoid of the nucleic acid sequence encoding a polynucleotide or a protein product thereof, characterized by being capable of inhibiting or reducing expression of the regulatory RNA polynucleotide of the second expression vector, an activity thereof, or both.

In some embodiments, the first expression vector further comprises a fourth nucleic acid sequence encoding a self-cleaving peptide sequence.

In some embodiments, the fourth nucleic acid sequence is located between the third nucleic acid sequence and the second nucleic acid sequence.

In some embodiments, the controlling comprises reducing high basal expression level of the GOI in the absence of the agent, preserving high expression level of the GOI in the presence of the agent, or both.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1C describe fold change activation (FCA) and block diagrams for indirect coherent feedforward (ICF) and double negative feedback (DNF) designs. (1A) Describes the transfer function of promoter activity. FCA is the ratio between ON and OFF states of the promoter. The OFF state is the minimum activity of the promoter and is achieved when no input molecules are present and there is only leaky gene expression (basal level) due to the unspecific binding of RNA polymerases (RNAPs). The ON state is the maximum activity of the promoter. “Th” is the input threshold of genetic switches and equals to half of FCA on the logarithmic scale. (1B) Schematic diagram of the ICF circuit. Input molecules regulate both the inhibitor and the output level. (1C) Schematic diagram of the mutual inhibition through DNF circuit. A positive feedback between the inhibitor and the output is coupled through mutual repression.

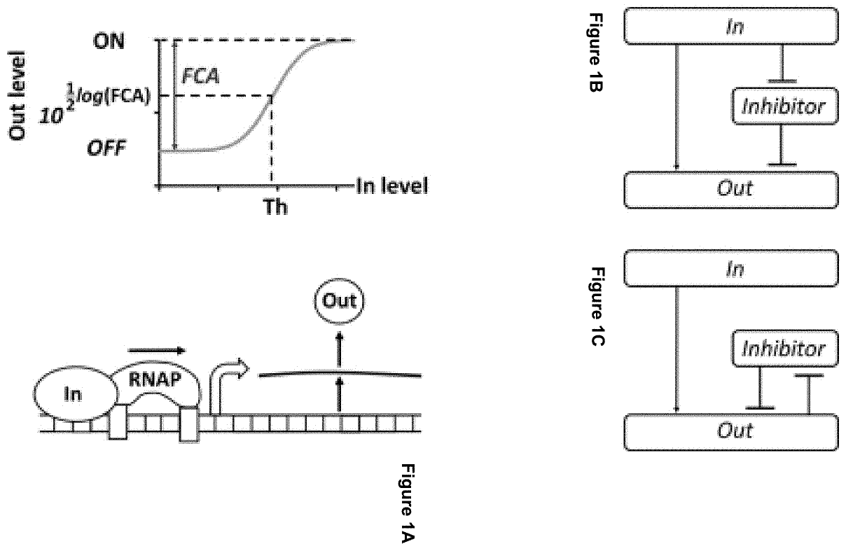

FIGS. 2A-2J depict linear models for ICF and DNF designs. (2A) Block diagram for the open loop (OL) circuit. The part under test (PUT) has a non-linear monotonic function with two distinct levels: (1) minimum level-β (e.g., basal level of promoter activity) and (2) normalized maximum level “1”. The output of the OL circuit is obtained by subtracting Fs from the output of the PUT. The connecting node has two inputs. One with a positive sign that is connected directly to the output of the PUT target circuit, and the second is with a negative sign that is connected to a constant “1” through a gain of Fs. (2B) Block diagram for the indirect coherent feedforward (ICF) circuit. The output of the PUT is split into two branches that both positively regulate the circuit output. The difference between the two branches determines the circuit output. The first branch is directly connected to circuit output and the second branch includes a two-stage subtraction with a gain of Fs. (2C) Block diagram for the mutual inhibition through double negative feedback (DNF) circuit. The output of the PUT is regulated through a negative feedback loop formed by an inverter (e.g., repressor) with gain of Fs. (2D) Simulation results of the OL circuit. (2E) Simulation results of the ICF circuit. (2F) Simulation results of the DNF circuit. (2G) FCA level versus FS strength for OL, ICF and DNF circuits. (2H) Maximum sensitivity versus FS strength for OL, ICF and DNF circuits. The sensitivity is calculated at every input point, based on S=dIyt/(Out)/dln/(in). (2I) Minimum detection level (MDL) versus FS strength for OL, ICF and DNF circuits. MDL is defined as the input level when the sensitivity is maximum. (2J) Qualitative β-FS diagrams for ICF and DNF designs. The area marked in light green corresponds to maximal FCA and the area marked in light yellow corresponds to minimal MDL. Outside these areas, the FCA and MDL are not optimal.

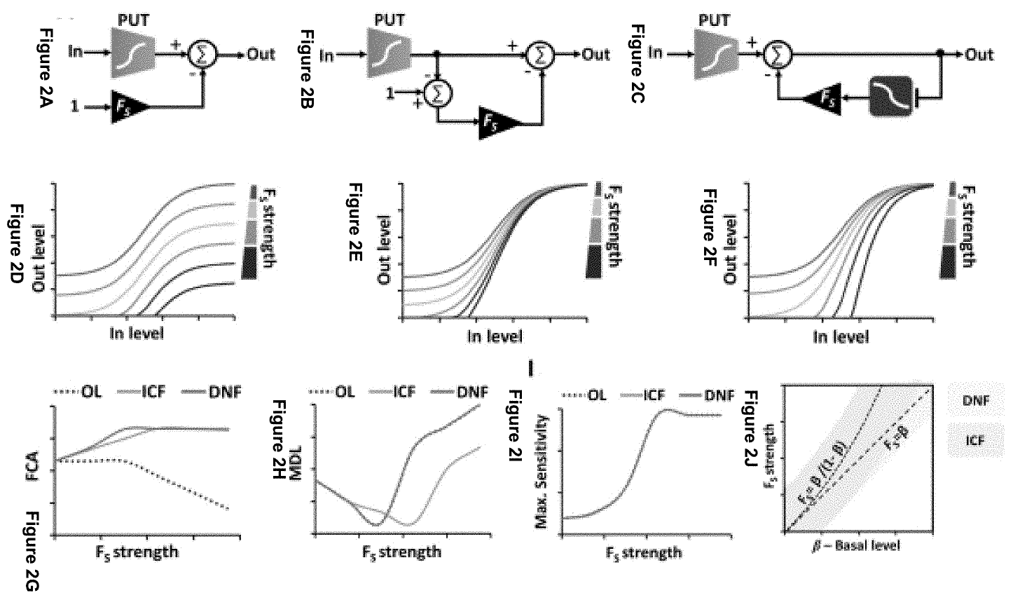

FIGS. 3A-3D depict models for ICF and DNF circuits based on biochemical reactions. (3A) Schematic diagram for molecular ICF network. Molecule Z is activated by molecule X and repressed by molecule Y, which is activated by X. (3B) Simulation results of FCA and MDL for molecular ICF circuit. (3C) Schematic diagram for molecular DNF circuit. Similar to the ICF, but here the molecule Z also represses Y. (3D) Simulation results of FCA and MDL for the molecular DNF circuit. Simulation parameters: β=0.1, α=10, n=1.5, m=1, h=1.

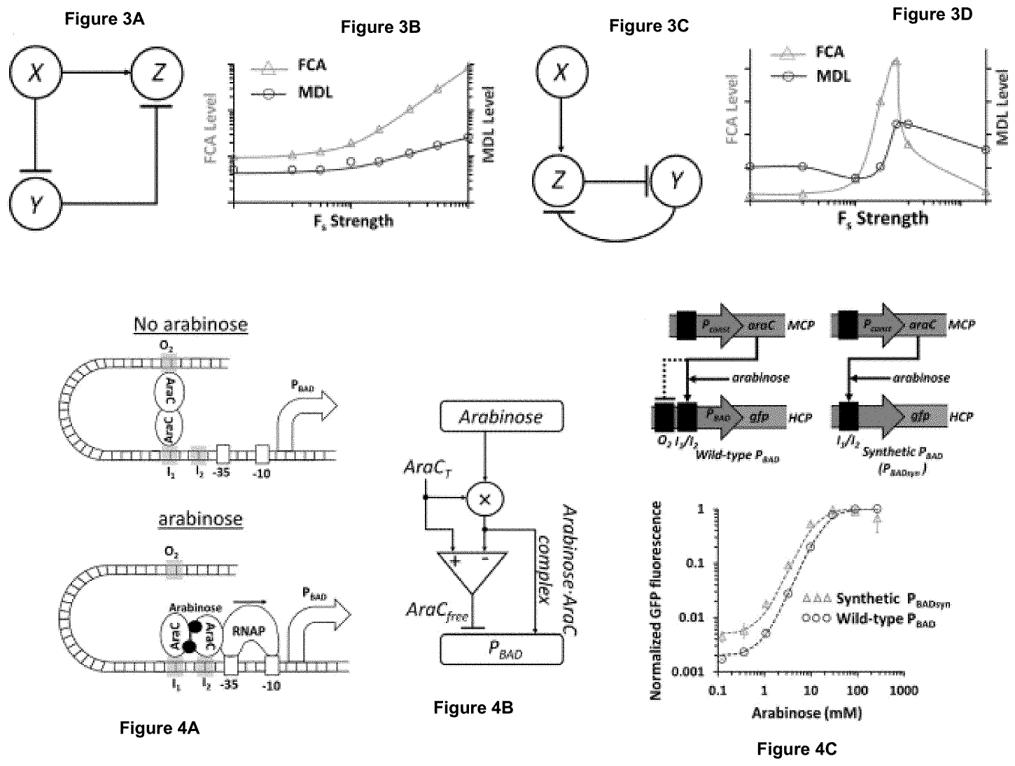

FIGS. 4A-4C depict ICF design describes L-arabinose utilization system. (4A) The structure of PBAD promoter in L-arabinose utilization system. In the absence of arabinose, a loop between O2 and I2 binding sites is formed through AraC, which prevents RNA polymerase from accessing the promoter. When arabinose is present, the loop is released and AraC binds to Ii and 12 sites. This leads to RNA polymerase (RNAP) binding to DNA sites (−35, −10) and the initiation of transcription. (4B) A diagram model for AraC and PBAD promoter showing that the system resembles an ICF network. On the one hand, the arabinose acts as an input to activate the PBAD by forming arabinose-AraC complex. On the other hand, the free AraC represses the PBAD promoter and is equal to the total concentration of AraC (AraCT) minus the arabinose-AraC complex concentration. (4C) The measured transfer function of wild-type PBAD and synthetic PBAD (PBADsyn). The synthetic PBADsyn contains only I₁ and I₂ binding sites without O₂ DNA sites. AraC is expressed by a constitutive promoter, encoded on a medium-copy-number plasmid (MCP). The synthetic PBADsyn and wild-type PBAD promoters regulate green fluorescent protein (GFP), encoded on a high-copy-number plasmid (HCP). The dotted lines are Hill function fittings. All experimental data are averaged from three experiments.

FIGS. 5A-5G depict implementation of ICF and DNF designs in living cells. (5A) Utilization of transcriptional interference to mimic subtraction. This design is similar to the open loop (OL) shown in FIG. 2A. The PPUT activates GFP signal. The Plux reverse promoter is located opposite to PPUT and upstream to gfp gene repressing GFP signal. The first unidirectional terminator is in the same orientation as PPUT and downstream to gfp gene. The second unidirectional terminator is in the same orientation as Plux and upstream to PPUT. The terminator is represented by a highlighted letter T. The RBS is marked by a blue rectangle. The riboj sequence is inserted upstream of the RBS which is marked by a circle. The LuxR transcription activator and mCherry are expressed under PtetO promoter, encoded on MCP. When no TetR is expressed, PtetO acts as a constitutive promoter. Both LuxR and mCherry genes have their own RBS sequences. The unit Terminator_RC-PPUT-Plux_RC¬¬GFP-Terminator is encoded on HCP. The block diagram describes the operation of OL circuit, where the output is regulated both by the input and inhibitor. (5B) Utilizing antisense transcription to mimic subtraction. This design is similar to OL in FIG. 2. The PPUT activates GFP signal. The Plux promoter is oriented in reverse to PPUT and downstream to gfp gene repressing GFP signal. The first unidirectional terminator was placed in the same orientation to PPUT and downstream to gfp gene. The second unidirectional terminator was placed in the same orientation to Plux and upstream to PPUT. The LuxR activator and mCherry are expressed by PtetO promoter encoded on MCP. Both LuxR and mCherry genes have their own RBS sequences. The unit Terminator_RC-PPUT-GFP-Plux_RC¬¬-Terminator is encoded on HCP. The block diagram describes the operation of OL, where both input and inhibitor regulate the output level. (5C) Implementation of an inverting switch using TetR repressor. The PPUT controls the expression of TetR, which represses the activity of PtetO. The small molecule aTc binds TetR to release the repression of PtetO. The PtetO-mCherry-Terminator construct was placed on MCP, while the PPUT-TetR-Terminator construct was cloned on LCP in order to match their copy numbers in ICF and DNF circuits. The mCherry gene was further replaced by LuxR gene to be integrated in ICF and DNF circuits. The block diagram describes the operation of inverting switch circuit. (5D) Implementation of ICF circuit by combining a transcriptional interference unit with TetR inverting switch. Here TetR is controlled only by PPUT. (5E) Implementation of a DNF circuit by combining transcriptional interference unit with TetR inverting switch. Here TetR is controlled by both PPUT and Plux promoters. (5F) Implementation of ICF circuit by combining an antisense transcription unit with TetR inverting switch. Here TetR is controlled only by PPUT. (5G) Implementation of a DNF circuit by combining an antisense transcription unit with TetR inverting switch. Here TetR is controlled by both promoters PPUT and Plux.

FIGS. 6A-6H depict transcriptional interference- based ICF and DNF topologies for synthetic PBADsyn and PlacO inducible promoters. (6A) Experimentally measured arabinose-GFP transfer function for the synthetic PBADsyn-based OL circuit as a function of AHL concentration. (6B) Experimentally measured arabinose-GFP and arabinose-mCherry transfer functions for the synthetic PBADsyn-based ICF circuit (TetR is fused with a LVA degradation tag) for various AHL concentrations. (6C-6E) FCA levels (6C), Maximum sensitivity level (6D), and MDL for different synthetic PBADsyn-based circuits (OL, ICF, DNF) versus AHL concentrations derived from experimental data (6E). (6F-6H) FCA levels (6F), Maximum sensitivity level (6G), and MDL for different PlacO-based circuits (OL, ICF) versus AHL concentration derived from experimental data (6H). The dotted lines are fittings using Hill-functions

$$( a \cdot \frac{{( AHL / {K}_{1} )}^{{n}_{1}}}{1 + {( AHL / {K}_{1} )}^{{n}_{1}}} \cdot \frac{1}{1 + {( \frac{AHL}{{K}_{1}} )}^{{n}_{1}}} + b ) .$$

All experimental data are averaged from three experiments.

FIGS. 7A-7H depict ICF and DNF topologies for specific bacterial biosensors sensitive to heme and arsenic (AsNaO2) based on antisense transcription. (7A) Blood sensor operation. Experimentally measured heme-GFP transfer function of a blood sensing circuit in the simplest (wild-type) design. Transporter proteins are constitutively expressed from ChuA gene. HrtR is a repressor and is driven by a constitutive promoter. A heme-group containing molecule enters the bacterial cells through the outer membrane ChuA protein and binds the transcriptional repressor HrtR to form a heme-HrtR complex which is then released from PLhrtO heme-inducible promoter allowing its activation and GFP expression. (7B) Experimentally measured heme-GFP transfer function of PLhrtO-based OL circuit relative to AHL concentration. (7C) The measured heme-GFP transfer function of PLhrtO based ICF circuit (TetR is fused with a LVA degradation tag) relative to AHL concentration. (7D) FCA levels derived from experimental results for various blood sensor circuits (OL, ICF, DNF) as a function of AHL concentration. (7E) Arsenic sensor circuit with inducible antisense transcription. The transcription factor ArsR encoded by arsR gene is constitutively expressed to repress ParsR promoter. Arsenic input, AsNaO₂, can bind with ArsR to release the repression on ParsR, to produce a GFP signal. A reverse Plux is located downstream of gfp gene to induce antisense transcription. The induction of antisense transcription is controlled by varying AHL concentrations. Experimentally measured arsenic-GFP transfer function of ParsR-based OL circuit under various AHL concentrations. (7F) Experimentally measured arsenic-GFP transfer function of ParsR-based ICF circuit (TetR is fused with a LVA degradation tag) under various AHL concentrations. (7G-7H) FCA levels (7G), and Maximum sensitivity derived from experimental results for various arsenic sensor circuits (OL, ICF, DNF) under various AHL concentrations (7H). The dotted lines are fittings using Hill-functions

$$( a \cdot \frac{{( AHL / {K}_{1} )}^{{n}_{1}}}{1 + {( AHL / {K}_{1} )}^{{n}_{1}}} \cdot \frac{1}{1 + {( \frac{AHL}{{K}_{1}} )}^{{n}_{1}}} + b ) .$$

All experimental data represent the average of three experiments.

FIGS. 8A-8J depict ICF and DNF designs for systemic bacterial biosensors based on oxidative stress response and SOS response. (8A) OL circuit based on antisense transcription for the katG biosensor inducible by hydrogen peroxide (H₂O₂). H₂O₂ interacts with the transcription factor OxyR causing a conformational change to its structure which in turn activates through a series of oxidative stress responses the PkatG promoter enabling GFP expression. OxyR is constitutively expressed. A reverse promoter, Plux, is placed downstream of PkatG as a transcriptional interference component, the strength of which can be programmed using AHL concentration. The experimentally measured H₂O₂-GFP transfer function of PkatG-based OL circuit under various AHL concentrations is also shown. (8B) Experimentally measured H₂O₂-mCherry transfer function of the inverting switch using TetR repressor. The PkatG promoter controls the expression of TetR, which represses the activity of PtetO. The small molecule, aTc, inhibits the activity of TetR. (8C) Experimentally measured H₂O₂-GFP transfer function of the PkatG-based DNF circuit (TetR is fused with a LVA degradation tag) under various AHL concentrations. (8D) FCA levels derived from experimental results for different katG biosensor circuits (OL, ICF, DNF) across AHL concentrations. (8E) OL circuit based on transcriptional interference for recA biosensor activated by Nalidixic Acid. The LexA repressor inhibits the activity of PrecA promoter, and Nalidixic Acid induces a series of SOS responses that inhibit the LexA activity. PrecA drives GFP expression and LexA is constitutively expressed. A reverse promoter, Plux, is placed downstream of PrecA as a transcriptional interference component, the strength of which can be programmed using AHL concentration. The experimentally measured Nalidixic Acid-GFP transfer function of PrecA-based OL circuit under various AHL concentrations, as well as the Nalidixic Acid-mCherry transfer function of PtetO in the absence of TetR are also shown. (8F) Experimentally measured Nalidixic Acid—GFP transfer function of PrecA-based ICF circuit (TetR is fused with a LVA degradation tag) using antisense transcription under AHL concentrations. The measured Nalidixic Acid-mCherry transfer function of PtetO-based ICF circuit is shown. (8G) and (8H) show FCA levels, Maximum sensitivity derived from experimental results for the OL and ICF circuits of recA biosensor across AHL concentrations. The dotted lines are fittings using Hill-functions

$$( a \cdot \frac{{( AHL / {K}_{1} )}^{{n}_{1}}}{1 + {( AHL / {K}_{1} )}^{{n}_{1}}} \cdot \frac{1}{1 + {( \frac{AHL}{{K}_{1}} )}^{{n}_{1}}} + b ) .$$

All experimental data represent the average of three experiments. (8I) A graph showing FCA levels. (8J) A graph showing maximal sensitivity.

FIGS. 9A-9G include representatives of distinct genetic circuits with the goal of lowering the basal level expression and keeping high expression of a target gene. (9A) RNA-only model. The gene of interest (GOT) is expressed under the inducer-regulated promoter under test (P_{PUT}). A binding-site sponge (BSS) is expressed under P_{PUT} as well. A small hairpin RNA (shRNA) is constitutively expressed on a fitting promoter. The shRNA can repress the expression of the GOI. However, the BSS creates a competition-derived repression of the shRNA repression. (9B) CRISPR model. GOI is expressed under PPUT. A catalytically dead clustered regularly interspaced short palindromic repeat interference (CRISPR)-associated protein (Cas) 9 fused to the Kruppel-associated box (KRAB) repressor (dCas9-KRAB) is expressed under the PPUT as well. A small hairpin RNA (shRNA) is constitutively expressed on a fitting promoter. The shRNA can repress the expression of the GOI. However, a small guide RNA (sgRNA) is also constitutively expressed. The sgRNA combines with the dCas9-KRAB to repress the expression of the shRNA, and in doing so represses its repression of the GOI. (9C) CRISPR-IRES model. GOI is expressed under P_{PUT}. The GOI is co-expressed with dCas9-KRAB, with an internal ribosome entry site (IRES) in between the two genes. A shRNA is constitutively expressed on a fitting promoter. The shRNA can repress the expression of the GOI. However, a sgRNA is also constitutively expressed. The sgRNA combines with the dCas9-KRAB to repress the expression of the shRNA, and in doing so represses its repression of the GOI. (9D) TF model. GOI is expressed under PPUT. A DNA-binding protein (GAL4) fused to the KRAB repressor (GAL4-KRAB) is expressed under the P_{PUT} as well. A shRNA is constitutively expressed on a fitting promoter. The shRNA can repress the expression of the GOI. However, the transcription factor fused with the repressor (GAL4-KRAB) can attach to binding sites at the promoter upstream to the shRNA, and in doing so represses its repression of the GOI. (9E) TF-IRES model. GOI is expressed under PPUT. The GOI is co-expressed with a DNA-binding protein (GAL4) fused to the KRAB repressor (GAL4-KRAB), with an IRES in between the two genes. A shRNA with a sequence matching part of the RNA sequence from the RNA sequence of the GOI is constitutively expressed on a fitting promoter. The shRNA is able to repress the expression of the GOI. However, the transcription factor fused with the repressor (GAL4-KRAB) is able to attach to binding sites at the promoter upstream to the shRNA, and in doing so represses its repression of the GOI. (9) Weak reverse promoter model. GOI is expressed under PPUT. The GOI is co-expressed with a DNA-binding protein (GAL4) fused to the KRAB repressor (GAL4-KRAB), with an IRES between the two genes. A weak reverse promoter (in the figure shown with minimal CMV promoter) with binding sites for the transcription factor (GAL4) is placed in a reverse formation downstream to the gene. This placement results in repression of the GOI and GAL4-KRAB. However, GAL4-KRAB can attach to binding sites at the weak reverse promoter, and in doing so represses its repression of the GOI. (9G) Intronic-shRNA model. GOI is expressed under PPUT. The GOI is co-expressed with a transcription factor (TF), with an intron and a self-cleaving peptide sequence (shown as P2A) in between the two genes. The intron acts like a shRNA. The intron can repress the expression of the GOI and the TF. However, the TF can attach to binding sites at the promoter upstream to a BSS which in turn is able to repress the repression by the intron shRNA.

### DETAILED DESCRIPTION

The present invention, in some embodiments thereof, is directed to systems comprising a plurality of expression vectors, as well as methods of using same, such as for, controlling expression level of a gene of interest (GOI) operably linked to an inducible promoter in a cell, or reducing high basal expression level of a GOI operably linked to an inducible promoter in the absence of an input signal while preserving high expression level of the GOI in the presence of the input signal.

The present invention, in some embodiments, is applicable in environmental monitoring for detection of toxic chemicals. Improving the FCA together with decreasing basal level expression could be integrated in applications for environmental monitoring for detection of toxic chemical to improve the sensitivity of cell-based biosensors to very low concentrations of target molecules.

The present invention, in some embodiments, is applicable in metabolic engineering, implementation of double negative feedback (DNF) designs into living cells can improve the balance of genes involved in metabolic pathways and ensure a high yield of the target products on the one hand and on the other hand it can downregulate the expression of toxic genes or products.

The present invention, in some embodiments, is applicable in therapeutics, improving the FCA and decreasing the leaky expression may improve safety of genetic circuits for cancer immunotherapy, gene therapy and other biotechnologies.

The present invention, in some embodiments, is applicable in medical diagnosis, for example in the detection of haematuria (a medical term for presence of blood in urine), which can be a serious medical condition. Another example is the detection of glucose in urine, where high glucose can be a sign for a health problem. The implementation of DNF design could improve the biosensing of blood or glucose in urine.

The present invention, in some embodiments, is applicable in cell-free systems (CFS), for improvement of biosensing in portable biosensors, such as biosensors for detection of mercury.

The present invention, in some embodiments, is applicable in cellular immunotherapy. The design could be incorporated within chimeric antigen receptor (CAR) T cell therapy that often shows ON target/OFF tumor toxicities to improve the efficiency and safety of the immunotherapy.

The present invention, in some embodiments, is applicable in virotherapy. The design could be incorporated within the viral genetic load to allow effective activation of transgenes within the specific appropriate target cell. Leakiness in the expression of the transgenes delivered via virotherapy hinders the efficacy of the treatment by either causing expression of genes in unwanted cells or diminishing the difference in expression between correct and incorrect target cells.

According to some embodiments, there is provided a system comprising at least 2 expression vectors, the system comprising: (a) a first expression vector comprising a first promoter sequence operably linked to a first nucleic acid sequence encoding a gene of interest (GOI) and a second nucleic acid sequence encoding a polynucleotide or a protein product thereof, characterized by being capable of inhibiting or reducing expression of a regulatory RNA polynucleotide, an activity thereof, or both, and (b) a second expression vector comprising a second promoter sequence operably linked to a nucleic acid sequence encoding the regulatory RNA polynucleotide.

In some embodiments, the system comprises a plurality of expression vectors. In some embodiments, a plurality comprises or refers to any integer equal to or greater than 2.

In some embodiments, at least 2 comprises: 2-3, 2-4, 2-5, 2-6, 2-7, 3-4, 3-5, 3-6, 4-5, 4-6, or 4-7. Each possibility represents a separate embodiment of the invention.

In some embodiments, the first promoter is responsive to an input signal.

In some embodiments, the second promoter is constitutively active. In some embodiments, the protein product characterized by being capable of inhibiting or reducing expression of a regulatory RNA polynucleotide, attaches to the binding site of the second promoter upstream to the nucleic acid sequence encoding the regulatory RNA polynucleotide, thereby repressing the expression of the nucleic acid sequence encoding the regulatory RNA polynucleotide. In some embodiments, the protein product characterized by being capable of inhibiting or reducing expression of a regulatory RNA polynucleotide, attaches to the binding site of the second promoter upstream to the nucleic acid sequence encoding the regulatory RNA polynucleotide, thereby repressing the expression of the nucleic acid sequence encoding the regulatory RNA polynucleotide and increasing, enhancing, preventing the repression, or any combination thereof, the expression of the GOI.

In some embodiments, the regulatory RNA polynucleotide inhibits or reduces expression levels of the GOI.

In some embodiments, the polynucleotide or a protein product thereof characterized by being capable of inhibiting or reducing expression of a regulatory RNA polynucleotide comprises a decoy protein-binding DNA site. In some embodiments, a decoy protein-binding DNA site comprises a binding-site sponge (BSS). In some embodiments, the polynucleotide or a protein product thereof characterized by being capable of inhibiting or reducing expression of a regulatory RNA polynucleotide comprises a DNA binding protein fused to a repressor. In some embodiments, the DNA binding protein comprises GAL4. In some embodiments, the repressor comprises KRAB. In some embodiments, the polynucleotide or a protein product thereof characterized by being capable of inhibiting or reducing expression of a regulatory RNA polynucleotide comprises GAL4 fused to KRAB (GAL4-KRAB).

In some embodiments, the first expression vector further comprises a third nucleic acid sequence encoding a protein translation regulatory element.

In some embodiments, the protein translation regulatory element increases, enhances, or both, the expression, stability, abundance, or any combination thereof, of the protein product of the GOI. In some embodiments, the protein translation regulatory element comprises or is an internal ribosome entry site (IRES).

In some embodiments, the regulatory RNA polynucleotide comprises or consists of an RNA interfering (RNAi) molecule.

Inhibitory nucleic acids useful in the present methods and compositions include antisense oligonucleotides, ribozymes, external guide sequence (EGS) oligonucleotides, siRNA compounds, single- or double-stranded RNA interference (RNAi) compounds such as siRNA compounds, modified bases/locked nucleic acids (LNAs), antagomirs, peptide nucleic acids (PNAs), and other oligomeric compounds or oligonucleotide mimetics which hybridize to at least a portion of the target nucleic acid and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof.

As used herein “an interfering RNA” refers to any double stranded or single stranded RNA sequence, capable—either directly or indirectly (i.e., upon conversion)—of inhibiting or down regulating gene expression by mediating RNA interference. Interfering RNA includes but is not limited to small interfering RNA (“siRNA”) and small hairpin RNA (“shRNA”). “RNA interference” refers to the selective degradation of a sequence-compatible messenger RNA transcript.

As used herein “an shRNA” (small hairpin RNA) refers to an RNA molecule comprising an antisense region, a loop portion and a sense region, wherein the sense region has complementary nucleotides that base pair with the antisense region to form a duplex stem. Following post-transcriptional processing, the small hairpin RNA is converted into a small interfering RNA by a cleavage event mediated by the enzyme Dicer, which is a member of the RNase III family.

A “small interfering RNA” or “siRNA” as used herein refers to any small RNA molecule capable of inhibiting or down regulating gene expression by mediating RNA interference in a sequence specific manner. The small RNA can be, for example, about 18 to 21 nucleotides long.

As used herein, an “antagomir” refers to a small synthetic RNA having complementarity to a specific microRNA target, with either mispairing at the cleavage site or one or more base modifications to inhibit cleavage. In another embodiment, an “antagomir” refers to a small synthetic RNA having complementarity to a population of microRNA targets, with either mispairing at the cleavage site or one or more base modifications to inhibit cleavage.

In some embodiments, the third nucleic acid sequence is located between the first nucleic acid sequence and the second nucleic acid sequence.

In some embodiments, the system further comprises a third expression vector. In some embodiments, the third expression vector comprises the first promoter sequence operably linked to a nucleic acid sequence encoding the polynucleotide or the protein product thereof, characterized by being capable of inhibiting or reducing expression of the regulatory RNA polynucleotide of the second expression vector, an activity thereof, or both.

In some embodiments, the first expression vector is devoid of the nucleic acid sequence encoding the polynucleotide or the protein product thereof, characterized by being capable of inhibiting or reducing expression of the regulatory RNA polynucleotide of the second expression vector, an activity thereof, or both.

According to some embodiments, there is provided a system comprising at least 3 expression vectors, the system comprising: (a) a first expression vector comprising a first promoter sequence operably linked to a first nucleic acid sequence encoding a GOI and a second nucleic acid sequence encoding a polynucleotide or a protein product thereof, characterized by being capable of inhibiting or reducing expression of a first regulatory RNA polynucleotide when complexed with a second regulatory RNA polynucleotide, an activity thereof, or both; (b) a second expression vector comprising a second promoter sequence operably linked to a nucleic acid sequence encoding the first regulatory RNA polynucleotide; and (c) a third expression vector comprising the second promoter sequence operably linked to a nucleic acid sequence encoding the second regulatory RNA polynucleotide.

In some embodiments, the second promoter is constitutively active. In some embodiments, a constitutive promoter comprises a U6 promoter (P_{U6}).

In some embodiments, the first regulatory RNA polynucleotide inhibits or reduces expression levels of the GOI.

In some embodiments, the polynucleotide or a protein product thereof characterized by being capable of inhibiting or reducing expression of a regulatory RNA polynucleotide comprises a nucleic acid sequence encoding a protein capable of binding to DNA. In some embodiments, a protein capable of binding to DNA comprises an enzyme. In some embodiments, a protein capable of binding to DNA comprises a CRISPR-associated protein (Cas). In some embodiments, the Cas is or comprises Cas9. In some embodiments, the Cas is or comprises a dead-Cas (dCas). In some embodiments, the dCas is or comprises dCas9. In some embodiments, the polynucleotide or a protein product thereof characterized by being capable of inhibiting or reducing expression of a regulatory RNA polynucleotide comprises dCas9 fused to KRAB (dCas9-KRAB).

In some embodiments, the first expression vector further comprises a third nucleic acid sequence encoding a protein translation regulatory element.

In some embodiments, the second regulatory RNA polynucleotide is characterized by being capable of inhibiting or reducing expression of the first regulatory RNA polynucleotide when complexed with the second nucleic acid sequence encoding a polynucleotide or a protein product thereof, characterized by being capable of inhibiting or reducing expression of a first regulatory RNA polynucleotide. In some embodiments, the second regulatory RNA polynucleotide is or comprises a single guide RNA (sgRNA).

In some embodiments, the system further comprises a fourth expression vector comprising the first promoter sequence operably linked to the nucleic acid sequence encoding the polynucleotide or a protein product thereof, characterized by being capable of inhibiting or reducing expression of the first regulatory RNA polynucleotide when complexed with the second regulatory RNA polynucleotide, an activity thereof, or both.

In some embodiments, the first expression vector is devoid of the nucleic acid sequence encoding a polynucleotide or a protein product thereof, characterized by being capable of inhibiting or reducing expression of the regulatory RNA polynucleotide of the second expression vector, an activity thereof, or both.

According to some embodiments, there is provided a system comprising an expression vector comprising: (a) a first promoter sequence operably linked to a first nucleic acid sequence encoding a GOI, a second nucleic acid sequence encoding a protein translation regulatory element, and a third nucleic acid sequence encoding a protein characterized by being capable of binding to a second promoter sequence; and (b) a second promoter.

In some embodiments, the protein characterized by being capable of binding to the second promoter sequence represses transcription from the second promoter.

In some embodiments, the second promoter is constitutively active.

In some embodiments, the second promoter is constitutively active, and transcribes in a direction opposite to the first promoter sequence.

In some embodiments, the second promoter is or comprises a cytomegalovirus promoter (CMV).

According to some embodiments, there is provided a system comprising: (a) a first expression vector comprising a first promoter sequence operably linked to a first nucleic acid sequence encoding a GOI, a second nucleic acid sequence encoding a transcription factor capable of activating transcription of a second promoter, and a third nucleic acid sequence encoding a first regulatory RNA polynucleotide capable of inhibiting or reducing expression levels of the GOI, the transcription factor, or both; and (b) a second expression vector comprising the second promoter sequence operably linked to a nucleic acid sequence encoding a second regulatory RNA polynucleotide.

According to some embodiments, there is provided a system comprising: (a) a first expression vector comprising a first promoter sequence operably linked to a first nucleic acid sequence encoding a first output protein/molecule, and a second promoter; and (b) a second expression vector comprising a third promoter operably linked to a second nucleic acid sequence encoding a first regulatory protein and a third nucleic acid sequence encoding a second output protein/molecule.

According to some embodiments, there is provided a system comprising: (a) a first expression vector comprising a first promoter operably linked to nucleic acid sequence encoding a regulatory protein; and (b) a second expression vector comprising a second promoter operably linked to a nucleic acid encoding an output molecule/protein.

According to some embodiments, there is provided a system comprising: (a) a first expression vector comprising a first promoter sequence operably linked to a first nucleic acid sequence encoding a first output protein/molecule, and a second promoter; (b) a second expression vector comprising a third promoter operably linked to a second nucleic acid sequence encoding a first regulatory protein and a third nucleic acid sequence encoding a second output protein/molecule; and (c) a third expression vector comprising a fourth promoter operably linked to a second regulatory protein.

According to some embodiments, there is provided a system comprising: (a) a first expression vector comprising a first promoter sequence operably linked to a first nucleic acid sequence encoding a first output protein/molecule, and a second promoter; (b) a second expression vector comprising a third promoter operably linked to a second nucleic acid sequence encoding a first regulatory protein; and (c) a third expression vector comprising a fourth promoter operably linked to a second regulatory protein.

In some embodiments, the second regulatory RNA polynucleotide inhibits or reduces expression of the first regulatory RNA polynucleotide of the first expression vector, an activity thereof, or both.

In some embodiments, the first expression vector further comprises a fourth nucleic acid sequence encoding a self-cleaving peptide sequence.

In some embodiments, the fourth nucleic acid sequence is located between the third nucleic acid sequence and the second nucleic acid sequence.

In some embodiments, the second promoter transcribes in a direction opposite to the first promoter sequence.

In some embodiments, the second promoter is located between the first promoter and the nucleic acid sequence encoding the first output protein/molecule.

In some embodiments, the second promoter is located 3′ to the nucleic acid sequence encoding the first output molecule/protein.

In some embodiments, the first expression vector comprises from 5′ to 3′ the first promoter, a nucleic acid sequence encoding the first output molecule, and the second promoter.

For a non-limiting example, first regulatory protein e.g., LuxR protein, as exemplified, modulates the second promoter, e.g., plux promoter, by binding to a region of the plux promoter.

In some embodiments, the second promoter is responsive to the first regulatory protein.

In some embodiments, the first expression vector further comprises at least one “regulatory sequence”. In some embodiments, the first expression vector further comprises a plurality of regulatory sequences. In some embodiments, the regulatory sequence or a plurality thereof regulate transcription of the first output molecule. In one embodiment, the regulatory sequence or plurality thereof regulate translation of an output molecule. In one embodiment, the regulatory sequence or plurality thereof regulate degradation of an output molecule. In some embodiments, the regulatory sequence or plurality thereof comprises a degradation tag. Non-limiting examples of a regulatory sequence include, but are not limited to, a ribosomal binding site (RBS), a riboswitch, a ribozyme, a guide RNA binding site, a microRNA binding site, a cis-repressing RNA, a siRNA binding site, and a protease target site. Regulatory sequences are typically located between a promoter and a nucleic acid to which it is operably linked such that the regulatory sequences is capable of regulating transcription and/or translation of the downstream (3′) nucleic acid and/or output molecule/protein. In some embodiments, a regulatory sequence or plurality thereof is located in the 5′ untranslated region (UTR) of a polynucleotide encoding an output molecule (e.g., gene or a transcript thereof). In some embodiments, a regulatory sequence or plurality thereof is located in the 3′ UTR of a nucleic acid and controls degradation of the nucleic acid. In some embodiments, a regulatory sequence or plurality thereof is transnationally-fused to a protein-coding sequence so as to affect stability and/or intracellular-localization of the protein.

In some embodiments, a regulatory sequence or plurality thereof comprises or is RBS. In some embodiments, the RBS is selected from: RBS30, RBS31, and RBS34.

In some embodiments, a regulatory sequence or plurality thereof comprises or is a ribozyme. In some embodiments is RiboJ.

In some embodiments, the system further comprises a third expression vector comprising the first promoter operably linked to a nucleic acid sequence encoding a second regulatory protein.

In some embodiments, the second promoter is responsive to or is induced by the second regulatory protein. In some embodiments, the second regulatory protein binds to the second promoter. In some embodiments, the second regulatory protein binds to the second promoter thereby inhibiting, blocking, or reducing the expression and/or transcription of the output molecule/protein.

In some embodiments, the third expression vector further comprises the second promoter. In some embodiments, the third expression vector further comprises a regulatory sequence or plurality thereof, as described herein.

In some embodiments, the third expression vector comprises the second promoter being located between the first promoter and the nucleic acid sequence encoding the second regulatory protein.

In some embodiments, the third expression vector comprises the second promoter being located 3′ to the nucleic acid sequence encoding the second regulatory protein.

In some embodiments, the third expression vector comprises from 5′ to 3′ the first promoter, a nucleic acid sequence encoding the second regulatory protein, and the second promoter.

In some embodiments, the second promoter is responsive to or is induced by the regulatory protein. In some embodiments, the regulatory protein binds to the second promoter. In some embodiments, the regulatory protein binds to the second promoter thereby inhibiting, blocking, or reducing the expression and/or transcription of the output molecule/protein.

In some embodiments, the regulatory protein is negatively regulated by a signal molecule, thereby is inhibited. In some embodiments, an inhibited regulatory protein does not inhibit, block, or reduce the expression and/or transcription of the output molecule/protein.

In some embodiments, the system further comprises a fourth expression vector comprising the first promoter operably linked to a third regulatory protein.

In some embodiments, the first regulatory protein modulates or controls the expression derived from or driven or by the second promoter of the first expression vector.

In some embodiments, the second regulatory protein modulates or controls the expression derived from or driven by the first promoter of the first expression vector.

In some embodiments, the third regulatory protein modulates or controls the expression derived from or driven by the fourth promoter of the third expression vector.

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for regulatory proteins, such as a transcriptional activator or repressor.

In some embodiments, the promoter is operably linked to a polynucleotide sequence, such as, but not limited to encoding a gene or polynucleotide of interest (i.e., targeted for expression).

The term “operably linked” is intended to mean that the polynucleotide sequence of interest is linked to the regulatory element or elements in a manner that allows for expression of the polynucleotide sequence (e.g., in an in vitro transcription/translation system, artificial cell, or in a host cell when the expression vector is introduced into the artificial or host cell).

In some embodiments, a promoter is considered “responsive” to an input signal if the input signal modulates the function of the promoter, indirectly or directly. In some embodiments, an input signal may positively modulate a promoter such that the promoter activates or increases, transcription of a nucleic acid to which it is operably linked. In some embodiments, an input signal may negatively modulate a promoter such that the promoter is prevented from activating or inhibits, or decreases, transcription of a nucleic acid to which it is operably linked. In some embodiments, an input signal modulates the function of the promoter directly by binding to the promoter or by acting on the promoter without an intermediate signal.

According to the present invention, in some embodiments thereof, a promoter responsive to an input signal and/or regulatory protein is considered an “inducible” promoter. Inducible promoters for use according to the present invention include any inducible promoter described herein or known to one of ordinary skill in the art. Non-limiting examples of inducible promoters include, but are not limited to, chemically-regulated, biochemically-regulated, and/or physically-regulated promoters, such as alcohol-regulated promoters, tetracycline-regulated promoters (e.g., anhydrotetracycline (aTc)-responsive promoters and/or other tetracycline-responsive promoter systems, which include a tetracycline repressor protein (tetR), a tetracycline operator sequence (tetO), and/or a tetracycline trans-activator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and/or light-regulated promoters (e.g., light responsive promoters from plant cells), or any combination thereof.

In some embodiments, the gene of interest may encode any protein which a skilled artisan wishes to produce (hereinafter “a polypeptide of interest”). In some embodiments, the polypeptide of interest is a full protein. In some embodiments, the polypeptide of interests is a fragment of a protein. In some embodiments, the polypeptide of interests is an enzyme. In some embodiments, the polypeptide of interests is an antibody. In some embodiments, the polypeptide of interests is a therapeutic protein. In some embodiments, the polypeptide of interest is a structural protein. In some embodiments, the polypeptide of interest is a scaffold protein. In some embodiments, the polypeptide of interest is a reporter gene. In some embodiments, the polypeptide of interest is a heterologous protein. In some embodiments, the polypeptide of interest is industrially relevant protein. Examples of industrial and pharmaceutically relevant proteins include, but are not limited antibodies, antibody fragments, hormones, interleukins, enzymes, coagulants and vaccines to name but a few. Specific examples of proteins include, but are not limited to, insulin, thyroid hormone, human growth hormone, follicle-stimulating hormone, factor VIII, erythropoietin, granulocyte colony-stimulating factor, alpha-galactosidase A, alpha-L-iduronidase, N-acetylgalactosamine-4-sulfatase, interferon, insulin-like growth factor 1, and lactase.

As used herein, the term “input signal” refers to any chemical (e.g., small molecule) or non-chemical (e.g., light or heat) signal in a cell, or to which the cell is exposed, that modulates, i.e., activates or inhibits, directly or indirectly, a component (e.g., a promoter) of a system as disclosed herein. In some embodiments, an input signal is a biomolecule that modulates the function of a promoter (referred to as direct modulation), or is a signal that modulates a biomolecule, which in turn modulates the function of the promoter (referred to as indirect modulation). In some embodiments, an input signal is endogenous to a cell or a normally exogenous condition, compound, protein, or any combination thereof, that contacts a promoter of a system as disclosed herein in such a way as to be active in modulating (e.g., inducing or repressing) transcriptional activity from a promoter responsive to the input signal (e.g., an inducible promoter).

Non-limiting examples of chemical input signals include, but not limited to, signals extrinsic or intrinsic to a cell, such as amino acids and amino acid analogs, saccharides and polysaccharides, nucleic acids, protein transcriptional activators and repressors, cytokines, toxins, petroleum-based compounds, metal containing compounds, salts, ions, enzymes, enzyme substrates, enzyme substrate analogs, hormones, quorum-sensing molecules, and others.

Non-limiting examples of non-chemical input signals include, without limitation, changes in physiological conditions, such as changes in pH, light, temperature, radiation, osmotic pressure, saline gradients, or any combination thereof.

In some embodiments, there is provided a cell comprising a system as disclosed herein.

In some embodiments, the cell is a transgenic cell. In some embodiments, the cell is a transformed cell. In some embodiments, the cell is a transfected cell.

In some embodiments, the cell is a prokaryotic cell. In some embodiments, the cell is a bacterial cell.

In some embodiments, the cell is a eukaryotic cell. In some embodiments, the cell is an animal cell. In some embodiments, the cell is a plant cell. In some embodiments, the cell is a mammalian cell.

As used herein, the terms “transgenic”, “transformed”, and “transfected” “cell” refers to any cell that has undergone human manipulation on the genomic or gene level. In some embodiments, the transgenic cell has had exogenous polynucleotide(s) and/or expression vector(s), such as disclosed herein, introduced into it. In some embodiments, a transgenic cell comprises a cell that has an artificial vector introduced into it. In some embodiments, a transgenic cell is a cell which has undergone genome mutation or modification. In some embodiments, a transgenic cell is a cell that has undergone CRISPR genome editing. In some embodiments, a transgenic cell is a cell that has undergone targeted mutation of at least one base pair of its genome. In some embodiments, the exogenous expression vector(s) (e.g., of a system as disclosed herein) is/are stably integrated into the cell. In some embodiments, the transgenic cell expresses a nucleic acid sequence of an expression vector of a system as disclosed herein. In some embodiments, the transgenic cell expresses a vector of the invention. In some embodiments, the transgenic cell expresses a gene of interest, or a polypeptide encoded therefrom.

Expressing of a gene within a cell is well known to one skilled in the art. It can be carried out by, among many methods, transfection, viral infection, or direct alteration of the cell's genome. In some embodiments, the gene is in an expression vector such as plasmid or viral vector. One such example of an expression vector containing p16-Ink4a is the mammalian expression vector pCMV p16 INK4A available from Addgene.

A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector or a poxviral vector. The promoters may be active in mammalian cells. The promoters may be a viral promoter.

In some embodiments, the vector is introduced into the cell by standard methods including electroporation (e.g., as described in From et al., Proc. Natl. Acad. Sci. USA 82, 5824 (1985)),Heat shock, infection by viral vectors, high velocity ballistic penetration by small particles with the nucleic acid either within the matrix of small beads or particles, or on the surface (Klein et al., Nature 327. 70-73 (1987)), and/or the like.

In some embodiments, nucleic acid sequences are transcribed by RNA polymerase II (RNAP II and Pol II). RNAP II is an enzyme found in eukaryotic cells. It catalyzes the transcription of DNA to synthesize precursors of mRNA and most snRNA and microRNA.

In some embodiments, mammalian expression vectors include, but are not limited to, pcDNA3, pcDNA3.1 (±), pGL3, pZeoSV2(±), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In one embodiment, plant expression vectors are used. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3:1671-1680 (1984); and Brogli et al., Science 224:838-843 (1984)] or heat shock promoters, e.g., soybean hsp17.5-E or hsp17.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide.

A person with skill in the art will appreciate that a gene can also be expressed from a nucleic acid construct administered to the individual employing any suitable mode of administration, described hereinabove (i.e., in vivo gene therapy). In one embodiment, the nucleic acid construct is introduced into a suitable cell via an appropriate gene delivery vehicle/method (transfection, transduction, homologous recombination, etc.) and an expression system as needed and then the modified cells are expanded in culture and returned to the individual (i.e., ex vivo gene therapy).

The term “expression profile” refers to expression of a group/set of genes. In some embodiments, the expression profile may be detected at the expression levels such as by analyzing and determining RNA values (e.g., mRNA or miRNA). The RNA levels may be determined in various samples including but not limited to cells, and exosomes. In some embodiments, the expression profile may be detected at the translation levels such as by analyzing and determining CTAs expressed on a cell surface such as by using antibodies.

As used in reference with the methods of the invention, “increase in expression of the expression profile” refers to a sum increase of expression of the specific set of CTAs. For a non-limiting example, a specific value of increase may be a result of increase of all the antigens of the set. Alternatively, specific value of increase may be a result of increase of only a few antigens of the set. In some embodiments, the increase refers to at least 10% increase, 20% increase, 30% increase, 40% increase, 50% increase, 60% increase, 70% increase, 80% increase, 90% increase, 100% increase in expression level of the expression profile.

A variety of known techniques may be suitable for determining an expression profile. Such techniques include methods based on hybridization analysis of polynucleotides and on sequencing of polynucleotides, and proteomics-based methods. In some embodiments, the determining step is performed by nucleic acid hybridization, nucleic acid amplification, or an immunological method. In some embodiments, the determining step is performed in-situ. In some embodiments, fluorescence labeling or staining are applied. In some embodiments, an imaging step is further applied.

In some embodiments, the expression profile is obtained by measuring protein levels of CT antigens. In some embodiments, the expression, and the level of expression, of proteins or polypeptides of interest can be detected through immunohistochemical staining of tissue slices or sections. Additionally, proteins/polypeptides of interest may be detected by Western blotting, ELISA or Radioimmunoassay (MA) assays employing protein-specific antibodies.

Alternatively, protein levels can be determined by constructing an antibody microarray in which binding sites comprise immobilized, preferably monoclonal, antibodies specific to a plurality of proteins of interest. Methods for making monoclonal antibodies are well known (see, e.g., Harlow and Lane, 1988, ANTIBODIES: A LABORATORY MANUAL, Cold Spring Harbor, N.Y., which is incorporated in its entirety for all purposes). In one embodiment, monoclonal antibodies are raised against synthetic peptide fragments designed based on genomic sequence of the cell. With such an antibody array, proteins from the cell are contacted to the array, and their binding is assayed with assays known in the art.

In some embodiments, the determining step comprises the step of obtaining nucleic acid molecules from a biological sample. In some embodiments, the nucleic acids molecules are selected from mRNA molecules, DNA molecules and cDNA molecules. In some embodiments, the cDNA molecules are obtained by reverse transcribing the mRNA molecules. In some embodiments, the expression profile is determined by measuring mRNA levels of CT antigens. Methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andres et al., BioTechniques 18:42044 (1995).

Numerous methods are known in the art for measuring expression levels of a one or more gene such as by amplification of nucleic acids (e.g., PCR, isothermal methods, rolling circle methods, etc.) or by quantitative in situ hybridization. Design of primers for amplification of specific genes is well known in the art, and such primers can be found or designed on various websites.

The skilled artisan will understand that these methods may be used alone or combined. Non-limiting exemplary method are described herein.

RT-qPCR: A common technology used for measuring RNA abundance is RT-qPCR where reverse transcription (RT) is followed by real-time quantitative PCR (qPCR). Reverse transcription first generates a DNA template from the RNA. This single-stranded template is called cDNA. The cDNA template is then amplified in the quantitative step, during which the fluorescence emitted by labeled hybridization probes or intercalating dyes changes as the DNA amplification process progresses. Quantitative PCR produces a measurement of an increase or decrease in copies of the original RNA and has been used to attempt to define changes of gene expression in cancer tissue as compared to comparable healthy tissues.

RNA-Seq: RNA-Seq uses recently developed deep-sequencing technologies. In general, a population of RNA (total or fractionated, such as poly(A)+) is converted to a library of cDNA fragments with adaptors attached to one or both ends. Each molecule, with or without amplification, is then sequenced in a high-throughput manner to obtain short sequences from one end (single-end sequencing) or both ends (pair-end sequencing). The reads are typically 30-400 bp, depending on the DNA-sequencing technology used. In principle, any high-throughput sequencing technology can be used for RNA-Seq. Following sequencing, the resulting reads are either aligned to a reference genome or reference transcripts or assembled de novo without the genomic sequence to produce a genome-scale transcription map that consists of both the transcriptional structure and/or level of expression for each gene. To avoid artifacts and biases generated by reverse transcription direct RNA sequencing can also be applied.

Microarray: Expression levels of a gene may be assessed using the microarray technique. In this method, polynucleotide sequences of interest (including cDNAs and oligonucleotides) are arrayed on a substrate. The arrayed sequences are then contacted under conditions suitable for specific hybridization with detectably labeled cDNA generated from RNA of a test sample. As in the RT-PCR method, the source of RNA typically is total RNA isolated from a tumor sample, and optionally from normal tissue of the same patient as an internal control or cell lines. RNA can be extracted, for example, from frozen or archived paraffin-embedded and fixed (e.g., formalin-fixed) tissue samples. For archived, formalin-fixed tissue cDNA-mediated annealing, selection, extension, and ligation, DASL-Illumina method may be used. For a non-limiting example, PCR amplified cDNAs to be assayed are applied to a substrate in a dense array. Microarray analysis can be performed by commercially available equipment, following manufacturer's protocols, such as by using the Affymetrix GenChip technology, or Incyte's microarray technology.

### Methods of Use

According to some embodiments, there is provided a method for controlling expression level of a gene of interest (GOI) operably linked to an inducible promoter in a cell.

According to some embodiments, there is provided a method for reducing high basal expression level of a GOI operably linked to an inducible promoter in the absence of an input signal while preserving high expression level of the GOI in the presence of the input signal.

In some embodiments, the promoter is responsive to an input signal. In some embodiments, the GOI expression is controlled by at least two negative feedback loops.

In some embodiments, the GOI expression is controlled by an indirect coherent feedforward loop.

In some embodiments, the method comprises contacting a cell as disclosed herein.

In some embodiments, the method comprises contacting a cell comprising a system as disclosed herein.

In some embodiments, the method comprises controlling the expression of GOI, an output molecule/protein or a plurality thereof, or any combination thereof, in a transgenic, transfected, or transformed cell, as disclosed herein, wherein the cell comprises the system disclosed herein.

In some embodiments, the method comprises contacting the cell with an effective amount of an agent triggering or providing an input signal, thereby controlling expression level of the GOI operably linked to an inducible promoter in a cell.

In some embodiments, the method comprising contacting a cell comprising the GOI operably linked to the inducible promoter, and having expression being controlled by at least two negative feedback loops, with an effective amount of an agent triggering or providing the input signal, thereby reducing high basal expression level of the GOI operably linked to an inducible promoter in the absence of an input signal while preserving high expression level of the GOI in the presence of the input signal.

In some embodiments, contacting comprises supplementing the cell with an effective amount of an agent triggering or providing the input signal.

In some embodiments, contacting comprises culturing the cell with or in the presence of an effective amount of an agent triggering or providing the input signal.

In some embodiments, controlling comprises reducing high basal expression level of the GOI in the absence of the agent, preserving high expression level of the GOI in the presence of the agent, or both.

In some embodiments, reducing comprises at least 5%, at least 15%, at least 25%, at least 50%, at least 65%, at least 75%, at least 85%, at least 95%, at least 97%, at least 99%, or 100% reducing, or any value and range therebetween. Each possibility represents a separate embodiment of the invention. In some embodiments, reducing comprises 5-50%, 10-90%, 20-99%, or 5-100% reducing. Each possibility represents a separate embodiment of the invention.

### General

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

In the description and claims of the present application, each of the verbs, “comprise”, “include”, and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” when combined with a value refers to plus and minus 10% of the reference value. For example, a length of about 1,000 nanometers (nm) refers to a length of 1,000 nm±100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

As used herein, the term “plurality” refers to any integer equal to or greater than 2.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B”.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, chemical and cell biology techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes Cellis, J. E., ed. (1994); “Culture of Animal Cells—A Manual of Basic Technique” by Freshney, Wiley-Liss, N.Y. (1994), Third Edition; “Current Protocols in Immunology” Volumes Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### Chemicals

Nalidixic acid (NA), hydrogen peroxide (H2O2), sodium (meta)arsenite (AsNaO2), hemin, arabinose, isopropyl-β-D-1-thiogalactopyranoside (IPTG), anhydrotetracyclin hydrochloride (aTc), and acyl homoserine lactone 3OC6HSL (AHL) were used as inducers and were obtained from Sigma-Aldrich.

### Bacterial Strains, Plasmids and Gene Origins

Standard molecular cloning techniques were used for plasmids construction. New England Biolab's (Beverly, Mass.) restriction endonucleases, Thermo Scientific FastDigest Restriction Enzymes, T4 DNA Ligase were used for plasmid construction. All plasmids in this work were built and transformed to NEB 10-beta Escherichia coli (araD139 D(ara-leu)7697 fhuA lacX74 galK (W80 D(lacZ)M15) mcrA galU recA1 endA1 nupG rpsL (StrR) D(mrr-hsdRMS-mcrBC). The PkatG, PrecA, and ParsR promoters were obtained by PCR amplification (Phusion High-Fidelity PCR Kit—New England Biolabs) from the genome of MG1655 E. coli (F—λ—ilvG—rfb-50 rph-1), with primers listed in Supplementary Information, Table S3 of Litovco et al., 2021 (Nucleic Acids Research, Volume 49, Issue 9, Pages 5393-5406). For part amplification from the genome, 5 ml of MG1655 strain E. coli were inoculated from frozen glycerol stocks and were grown for 16 hours. The next morning, 5 μl from the overgrown culture was mixed with 15 μl of DNase and RNase free water, heated at 96° C. for 6 min and incubated at −80° C. for 10 min. Two (2) μl from this solution was added into PCR mixture with total volume of 50 μl. The primers were synthesized by Integrated DNA Technologies (Leuven, Belgium). Plasmids for cloning were transformed into chemically competent E. coli 10-beta with a standard heat shock protocol. Bacterial cultures were consistently cultured at 37° C. in Luria-Bertani (LB) Broth, Miller (Difco). The overnight grown cells were grown from glycerol stocks in 5 ml at 37° C. or inoculated from colonies on agar plate with appropriate antibiotics for plasmid preparation in the next morning. The growth media was supplemented with appropriate concentration of antibiotics: carbenicillin (50 μg/ml), kanamycin (30 μg/ml), or/and chloramphenicol (34 μg/ml). Plasmids were extracted from the bacterial cells with QIAprep Spin Miniprep Kit (Qiagen, Hilden, Germany) according to the manufacturer's manual. Colony screening was carried out by PCR with suitable forward and reverse primers. Sequencing was approved by Macrogen Sequencing Service (Macrogen Europe, the Netherlands). All synthetic parts used in this work are listed in Supplementary Information, Table S2 of Litovco et al., 2021 and the plasmid maps are included in Supplementary Information sections 5 and 6 of Litovco et al., 2021.

### Plasmid Construction

All plasmids in this work were constructed in a similar manner: Promoter-RB 5-gene-terminator-origin-of-replication-antibiotic-resistance, where the origin-of-replication was changed with AvrII and Sad restriction enzymes, the gene was replaced with restriction enzymes Kpnl and BamHI, and the antibiotic resistance was replaced with SacI and AatII restriction enzymes. Different combinations of plasmids forming different synthetic circuits (are summarized in Supplementary Information, Table S1 of Litovco et al., 2021) were transformed into competent E. coli 10-beta or MG1655 . coli either through heat-shock protocol or electroporation protocol.

### Cytometry Measurement and Data Analysis

Different combinations of plasmids forming different synthetic circuits were transformed into competent NEB 10-beta E. coli for cytometry measurements except from PLhrtO and PrecA, which were transformed into MG1655 E. coli wild type strain. The bacterial cultures were inoculated from colonies on agar plate the previous day and grown in 5 ml of LB with appropriate antibiotics at 37° C. and 300 r.p.m. In the morning, the overnight grown bacterial cultures were diluted 1:100 into fresh LB medium (for PlacO, PLhrtO, and ParsR circuits) or were diluted 1:50 into fresh M9 minimal media (1× M9 Salts (Sigma-Aldrich, M6030), 2 mM MgSO₄, 100 μM CaCl₂, 0.4% glucose, 0.1% casamino acids, 50 mg/1 thiamine) for the cytometry experiment with appropriate concentration of antibiotics (for PBAD, for PBADsyn, PkatG and PrecA) and incubated for specific time for regrowth and adaptation in fresh media, as described in section 3 for each promoter. Bacterial cultures were transformed into 96-well plates with known concentrations of inducers to total volume of 200 μl, incubated in microplate shaker (37° C., 500 r.p.m) for relevant time described in section 3 for each promoter until they reached optical density OD600 nm ˜0.4-0.7. Then, the fluorescence and scattering of bacterial cultures were analysed through flow cytometry analyzer (CytoFLEX S Flow Cytometer). In all experiments 10,000 events have been obtained and the fluorescence and forward and side scattering were taken using CytExpert 2.2 software. The fluorescence distribution data over population data were extracted together with its geometric mean from each well in 96-well plate and plotted using MATLAB. Fluorescence measurement was based on geometric mean of flow cytometry populations from three experiments. The flow cytometry data for one representative experiment for each combination, which was independently repeated for two more times, is provided in Supplementary Information, section 4 of Litovco et al., 2021. Next, the figures were built in EXCEL, based on geometric mean of flow cytometry populations with error bars representing the standard deviation errors of the geometric mean.

### EXAMPLE 1

### Indirect Coherent Feedforward (ICF) in Natural Biological Systems

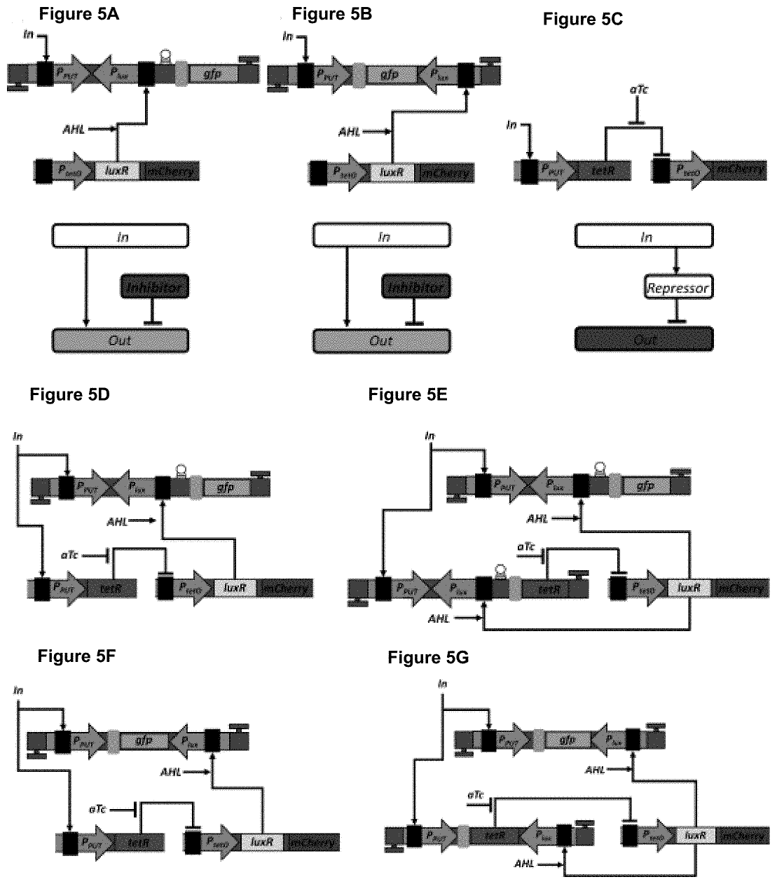

The inventors started the current study by searching for natural biological systems that contain ICF and DNF designs. The inventors found that the ICF network naturally occurs in the L-arabinose utilization system (FIG. 4A). In the absence of arabinose, AraC protein binds araI₁(I₁) and araO₂ (O₂) DNA binding sites by rigidly holding the DNA binding domains through its N-terminal arm. This conformation creates a loop in the DNA that prevents the RNA polymerase from binding to initiate transcription. When arabinose is present, the physically closed loop is released and AraC moves to bind aralI₁ (I₁) and araI₂ (I₂) DNA binding sites. The opened loop allows RNA polymerase to freely access the promoter, and the positioning of a DNA binding domain of AraC at I₂ facilitates transcription initiation by RNA polymerase. According to this explanation, a diagram model that describes the P_{BAD} promoter system is shown in FIG. 4B. While the arabinose—AraC complex activates the P_{BAD} promoter, the free AraC represses the P_{BAD} promoter. Since the total amount of AraC is equal to the amount of free AraC and that of the arabinose-AraC complex, in the herein disclosed model, the arabinose participates in two circuit branches. The first is driven by the complex (arabinose-AraC) which directly activates the output. The second branch indirectly activates the output through double inhibitions: the free AraC and what resides from the complex. Motivated by this model, the inventors modified the wild-type P_{BAD} that has I₁/I₂ and O₂ binding sites, by removing O₂ binding sites (FIG. 4C). The new synthetic P_{BADsyn} promoter has only I₁/I₂ binding sites. Thus, in the absence of arabinose, RNA polymerase can bind to the P_{BADsyn} promoter, leading to higher leaky gene expression than the wild-type P_{BAD} promoter (FIG. 4C).

### EXAMPLE 2

### Implementation of ICF and DNF Designs

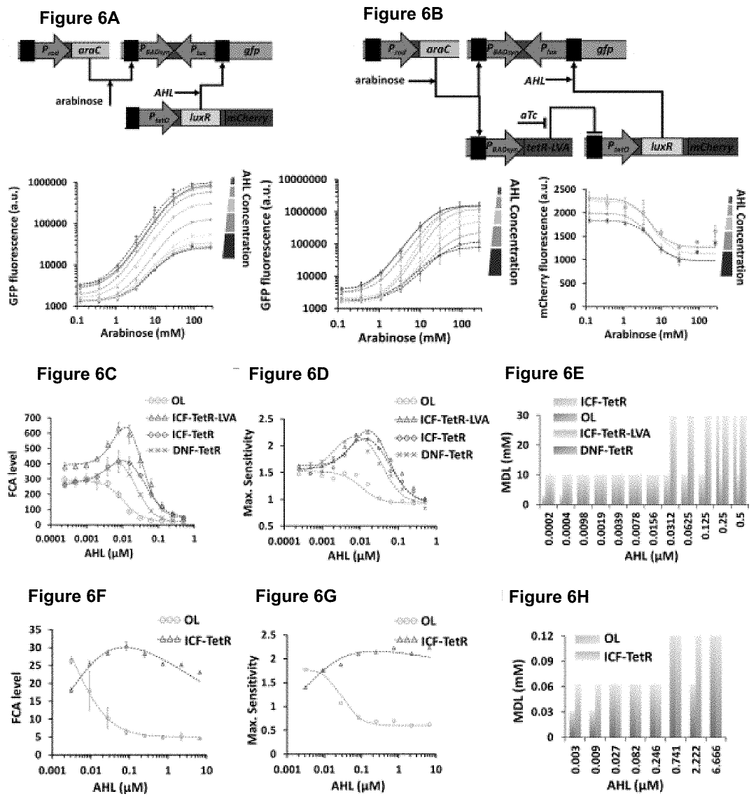

The inventors started to implement the ICF and DNF designs in living cells by mimicking a subtraction using a transcriptional interference system and an antisense transcription system. In both systems, the inventors placed the Pₗᵤₓ promoter in opposite orientation to promoter under test (P_{PUT}), which inhibits the P_{PUT} activity. The inventors started with the transcriptional interference system (See FIG. S5A of Litovco et al., 2021) that involves P_{PUT} followed by a downstream transcriptional-regulation component. The interference component, Pₗᵤₓ promoter, is oriented in the opposite direction to P_{PUT} and located upstream to the gfp gene. Thus, in such a system, the output GFP signal is activated by P_{PUT} promoter and is repressed by transcription of the reverse Pₗᵤₓ promoter. This special organization allows interference between forward and reverse promoters due to collisions of RNAPs actively transcribing from these promoters. The second system is the antisense transcription (See FIG. S5B of Litovco et al., 2021) that also involves a P_{PUT} and an interference component from Pₗᵤₓ promoter. The Pₗᵤₓ promoter is oriented oppositely to P_{PUT} and is located downstream to gfp gene. Consequently, the output GFP signal is activated by P_{PUT} promoter and is repressed by the antisense (reverse) Pₗᵤₓ promoter, where both DNA strands are fully transcribed in both directions to produce sense mRNA and antisense RNA. This special organization allows interference between forward and reverse promoters due to direct interaction between mRNA and antisense RNA. In this study, the inventors successfully built a protocol including five steps that guarantees an improvement in the FCA of P_{PUT}. In the first two steps the inventors characterized the behavior of the transcriptional interference unit (FIG. 5A) and antisense transcription unit (FIG. 5B) by varying acyl homoserine lactone (AHL) concentration. The AHL binds to transcription factor LuxR and forms a complex which activates the transcription of Pₗᵤₓ promoter. Thus, by varying AHL concentration, the inventors can control the strength of the feedforward/feedback loop (Fₛ). In the third step, the inventors implemented an inverting switch through TetR repressor (FIG. 5C) which is regulated by P_{PUT}. The inventors can tune P_{tetO}/TetR behavior either by changing TetR level (fusion with different ssrA degradation tags) or by varying anhydrotetracycline (aTc) concentration. After selecting an optimal aTc concentration, which gives the highest ON/OFF ratio of LuxR levels represented by mCherry, the inventors can implement the ICF and DNF gene circuits. Then, the inventors combine the transcriptional interference unit (FIGS. 5D-5E) or antisense transcription unit (FIGS. 5F-5G) with the inverting switch as described in step three. The difference between ICF and DNF implementations is that in the ICF design, TetR is regulated only by P_{PUT} and GFP is regulated by P_{PUT} and Pₗᵤₓ, whereas in the DNF design, both proteins, GFP and TetR are regulated by P_{PUT} and Pₗᵤₓ. Based on the genetic circuits shown in FIG. 5, the inventors modified the three-nodes molecular models to create genetic four-nodes models. The new models showed that in both ICF and DNF circuits an optimum FCA level is achieved when Fₛ increases (See supplementary Information, Section 2.1 and 2.2 of Litovco et al., 2021).

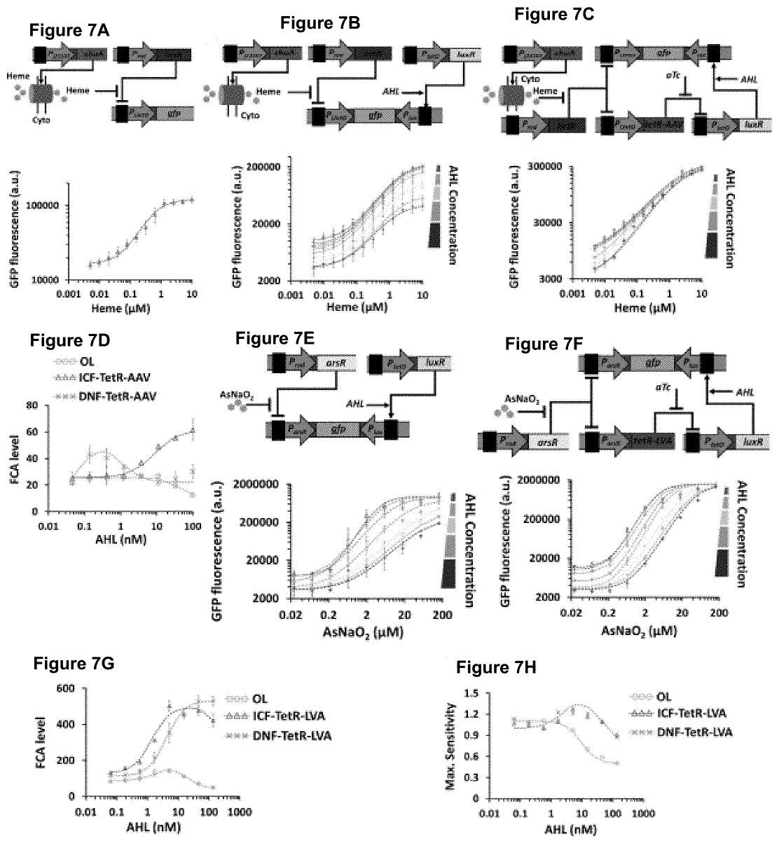

According to the herein disclosed simulation results (FIG. 5, and FIGS. S12-S16 of Litovco et al., 2021), the inventors tested the L-arabinose regulation system with the new synthetic P_{BADsyn} promoter (without the O₂ DNA binding site, FIG. 4). The inventors constructed the ICF and DNF circuits based on P_{BADsyn} using the transcriptional interference model. In the OL circuit both basal and maximum levels decrease as AHL increases (FIG. 6A). In the ICF circuit on the other hand, at a specific value of AHL (7.8×10⁻³ μM) the basal level decreases to very low values, while the maximum level only slightly decreases (FIG. 6B). The herein disclosed experimental results also show that TetR acts as an “Inverter-logic-gate” to control the LuxR expression. Other topologies of synthetic gene circuits, such as ICF and DNF containing TetR repressor without the degradation tag, were considered and constructed for optimizing the FCA of P_{BADsyn} (See supplementary Information, Section 3, FIG. S17 of Litovco et al., 2021). The FCA levels based on the experimental results for the P_{BADsyn} P circuit are shown in FIG. 6C. All circuits, except for the OL, showed an optimal FCA and maximum sensitivity (FIG. 6D) as a function of AHL concentration. The inventors also derived MDL (FIG. 6E) from sensitivity values for various circuits (FIG. S18 of Litovco et al., 2021). At the AHL concentration yielding the highest FCA level, the MDL is very low. In conclusion, for different design topologies an appropriate F_{S} strength allows FCA level to be improved up to three times (from ON/OFF=215 to ON/OFF=630) without compromising the MDL. The inventors also experimentally tested P_{lacO} promoter with an isopropyl β-D-1-thiogalactopyranoside (IPTG) inducer using the transcriptional interference model. The OL and ICF circuits showed consistent behaviors with our mathematical models (FIGS. 6F to 6H). Further analysis of the P_{lacO} construct is provided in Supplementary Information, Section 3.2 (FIG. S19 of Litovco et al., 2021).

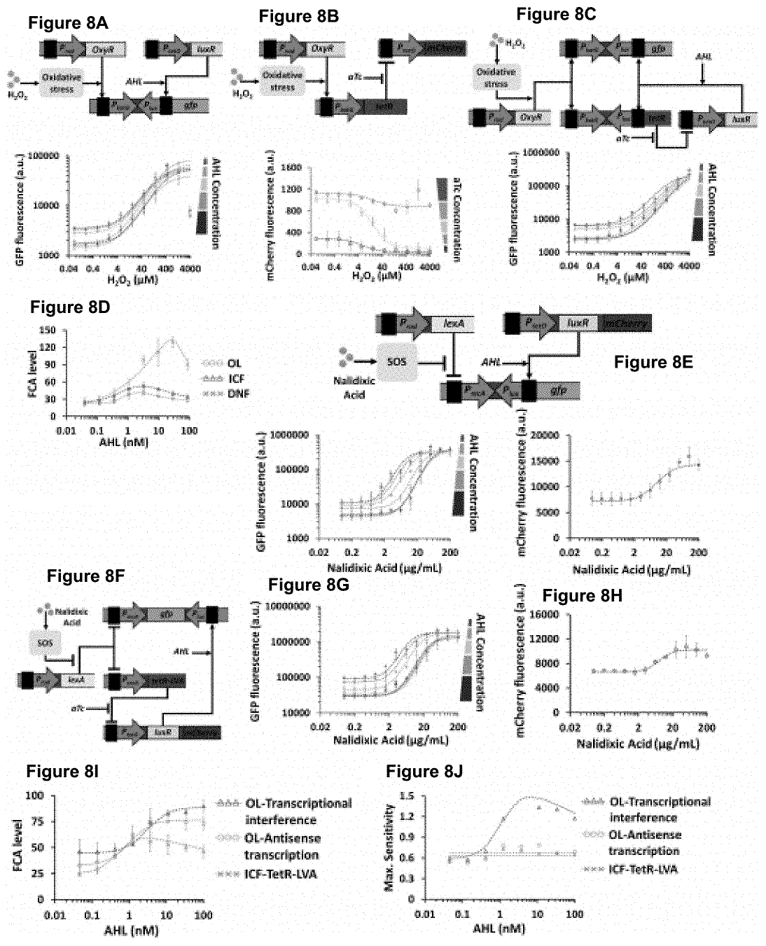

As an application the inventors used ICF and DNF designs to improve the performance of different types of bacterial biosensors, specifically for detection of heme, arsenic, hydrogen peroxide, and Nalidixic Acid toxins. Heme is released from lysed red blood cells, and the presence of this biomolecule in clinical samples is indicative of bleeding. The heme biosensor consists of three synthetic parts (FIG. 7A); the ChuA protein, the HrtR repressor and the synthetic P_{LHrtO} promoter. ChuA is an outer-membrane transporter from Escherichia coli strain O157:H7 that facilitates heme entry across cellular membranes. The HrtR repressor inhibits P_{LHrtO} promoter activity. A Heme-containing molecule binds to HrtR forming the complex Heme-HrtR which is released from P_{LHrtO} promoter allowing its activation with FCA≈7 (FIG. 7A). The OL circuit of the heme sensor based on antisense transcription reduced both the basal and maximum levels across the whole AHL range acting as a subtractor (FIG. 7B). In contrast, both ICF (FIG. 7C) and DNF (FIG. S20E of Litovco et al., 2021) circuits designed for heme detection in combination with antisense transcription units reduced the basal level without decreasing the maximum level. At specific AHL concentrations, FCA can be increased to 60 and 40 in ICF and DNF respectively, as shown in FIG. 7D. While the DNF circuit reaches an optimal FCA level, the FCA level of the ICF circuit monotonically increases as AHL concentration increases. Further analysis of the heme biosensor is provided (See supplementary Information, Section 3.3, and FIG. S20 of Litovco et al., 2021).

Arsenic is a heavy metal, which can contaminate drinking water and its long-term exposure can lead to toxicity and health issues including skin diseases and cancer. The Arsenic biosensor has an ArsR repressor and a synthetic promoter (P_{arsR}). In the wild-type circuit (FIG. S21A of Litovco et al., 2021), ArsR binds P_{arsR} forming the Arsenic-ArsR complex which is released from P_{arsR} promoter allowing its activation. In the OL circuit, increasing AHL concentration decreases both basal and maximum levels (FIG. 7E), whereas in ICF (FIG. 7F) and DNF (FIG. S21E of Litovco et al., 2021) circuits only the basal level is reduced, resulting in maximal FCA≈500 for both circuits. The FCA levels derived from the experimental results for arsenic biosensor are shown in FIG. 7G. The ICF circuit shows an optimal FCA. Interestingly, the OL circuit also demonstrates an optimal FCA that is in good agreement with our models (Supplementary Information Section 2.1, and FIG. S10 of Litovco et al., 2021). Such behavior can be obtained when the reverse promoter Pₗᵤₓ affects the switching threshold of the forward promoter. The FCA level of the DNF circuit monotonically increases as a function of AHL concentration. For the ICF, the maximum sensitivity (˜1.3) and maximum FCA level (˜500) were obtained at the same AHL (˜4 nM), while for the DNF, the maximum sensitivity and maximum FCA levels were obtained at different AHL concentrations (FIG. 7H, AHL˜4 nM for maximum sensitivity and AHL˜150 nM for maximum FCA). Further analysis for the arsenic biosensor is provided in Supplementary Information, Section 3.4, and FIG. S21 of Litovco et al., 2021. In addition, the behavior of P_{arsR}—based ICF circuit remained stable over the course of approximately eleven hours (FIG. S21J of Litovco et al., 2021).

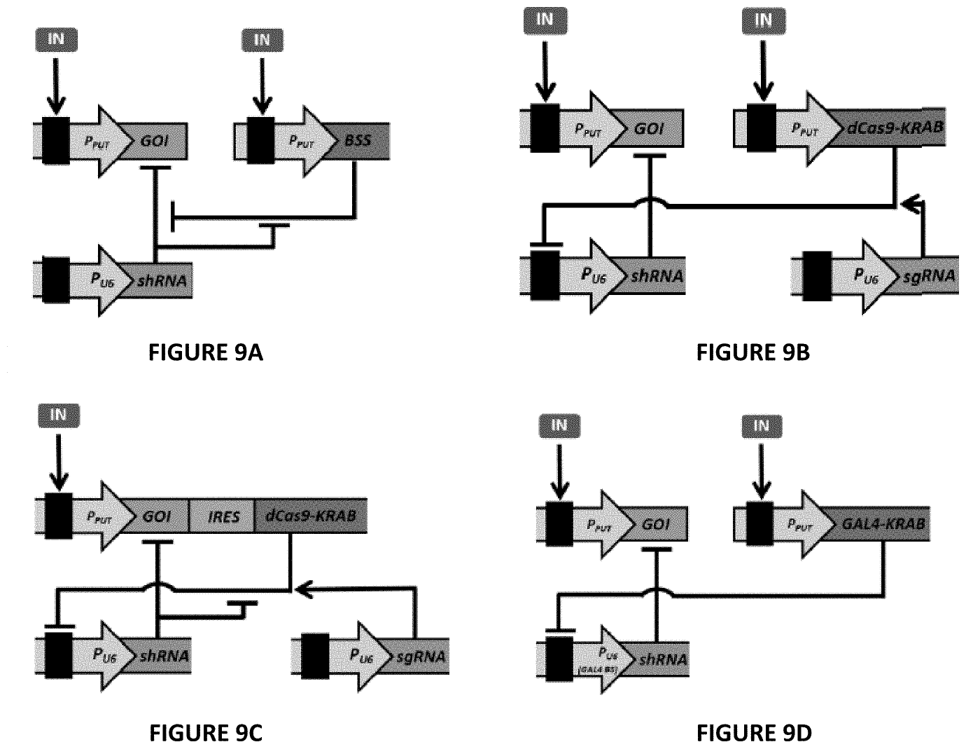

So far, the inventors have applied the proposed designs (OL, ICF and DNF) in biological systems where chemical inputs directly interact with target promoters by binding transcription factors. However, biological systems often activate multiple pathways in response to chemical signals and more specifically in response to toxic chemicals. For example, cells induce repair systems by activating cascades of regulators, e.g. oxidative stress response and SOS response. Implementing the ICF and DNF designs in these complex biological systems can be challenging from a circuit design aspect. To further explore the applicability of the herein disclosed designs, the inventors first examined the oxidative stress response that is sensitive to hydrogen peroxide (H₂O₂). The transcriptional regulator, OxyR, is activated by oxidation of H₂O₂ which in turn activates several genes involved in bacterial defense mechanisms, among them is the KatG gene. In the wild-type circuit, the transcriptional activator OxyR binds the katG promoter (P_{katG}) allowing its activation with FCA level of 10 (FIG. S22A of Litovco et al., 2021). The inventors implemented the OL circuit based on transcriptional interference as shown in FIG. 8A. The OL circuit reduced the basal and maximum levels across the entire AHL range with an increase in the switching threshold. The current experimental results also show that TetR acts as an “Inverter-logic-gate” to control LuxR expression level (FIG. 8B). At a specific AHL concentration, the DNF circuit reduced P_{katG} basal level without affecting the maximum levels (FIG. 8C). The FCA levels under various AHL concentrations are derived from the experimental results for katG biosensors and are shown in FIG. 8D. For OL, ICF and DNF circuits, FCA reaches optimal values when AHL concentrations fall between 1 to 10 nM, with increasing MDL (FIG. S22 of Litovco et al., 2021). Further analysis of the katG biosensors is provided in Supplementary Information, Section 3.5 of Litovco et al., 2021.

The SOS response in cells is induced by the DNA damage repair process and involves the activation of more than 40 proteins, including recA gene and its transcriptional repressor LexA. To build bacterial biosensors that are sensitive to SOS response, first, a synthetic circuit that includes recA promoter (P_{recA}) and LexA repressor was integrated into bacterial cells. The circuit is induced by Nalidixic Acid toxin and demonstrated a FCA level of 50 (FIG. S23A of Litovco et al., 2021). In such circuit, there is no direct interaction between Nalidixic Acid and LexA. Then, the inventors applied the OL design with recA promoter using both transcriptional interference (FIG. 8E) and antisense transcription (FIG. S23B of Litovco et al., 2021). The OL designs reduced the basal and maximum levels of P_{recA} across the entire range of AHL concentration and increased the switching threshold. In the case of P_{rec} promoter, the activity of P_{tetO} promoter in the absence of TetR (P_{tetO} is effectively a constitutive promoter) exhibits a strong dependency on Nalidixic Acid concentrations (mCherry signal in FIG. 8E, and in FIG. S23B of Litovco et al., 2021). Such a dependency has not been observed with other inducers when applied to P_{BAD}, P_{lacO}, P_{LhrtO}, P_{arsR}, and P_{KatG}. The dependency of P_{tetO} activity on Nalidixic Acid also affected the behavior of the ICF circuit (FIG. 8F). For example, as P_{tetO} is supposed to act as an inverter gate where the mCherry signal decreases as a function of Nalidixic Acid, instead, the mCherry signal increases when the Nalidixic Acid concentration is above 2 μg/ml. This trend in P_{tetO} activity affects the optimal FCA level of the circuits. The FCA and maximum sensitivity levels derived from the experimental results for the recA biosensors are shown in FIGS. 8G and 8H, respectively. The OL design performs best among the three designs, showing a doubled FCA level and maximum sensitivity. Further analysis of the recA biosensor is provided in Supplementary Information, Section 3.6, and FIG. S23 of Litovco et al., 2021.

### EXAMPLE 3

### Genetic Circuits

The inventors have constructed 7 distinct genetic circuits with the goal of lowering the basal level expression and keeping high expression of a target gene, resulting in an improvement in the fold change activation of the gene downstream to the promoter under test (P_{PUT}).

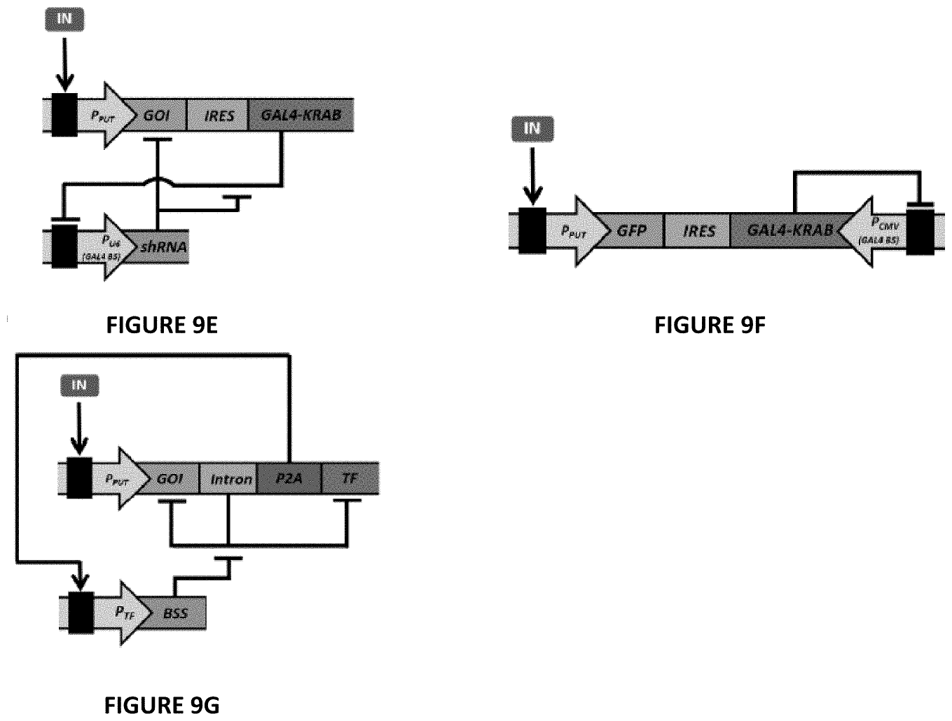

In the first genetic circuit (FIG. 9A), the gene of interest (GOT) is expressed under the inducer-regulated promoter under test (P_{PUT}). A binding-site sponge (BSS) is expressed under PPUT as well, be it on the same DNA fragment on a separate one. A small hairpin RNA (shRNA) with a sequence matching part of the RNA sequence from the RNA sequence of the GOI is constitutively expressed on a fitting promoter. The shRNA is able to repress the expression of the GOI. However, the BSS includes multiple repeats of the same matching RNA sequence, creating a competition-derived repression of the shRNA repression.

In the second genetic circuit (FIG. 9B), the GOI is expressed under P_{PUT}. A catalytically dead clustered regularly interspaced short palindromic repeat interference (CRISPR)-associated protein (Cas) 9 fused to the Kruppel-associated box (KRAB) repressor (dCas9-KRAB) is expressed under the PPUT as well, be it on the same DNA fragment on a separate one. A small hairpin RNA (shRNA) with a sequence matching part of the RNA sequence from the RNA sequence of the GOI is constitutively expressed on a fitting promoter. The shRNA is able to repress the expression of the GOI. However, a small guide RNA (sgRNA) with a sequence fitting the promoter upstream to the shRNA is also constitutively expressed. The sgRNA combines with the dCas9-KRAB to repress the expression of the shRNA, and in doing so represses its repression of the GOI.

In the third genetic circuit (FIG. 9C), the GOI is expressed under PPUT. The GOI is co-expressed with dCas9-KRAB, with an internal ribosome entry site (IRES) in between the two genes. A shRNA with a sequence matching part of the RNA sequence from the RNA sequence of the GOI is constitutively expressed on a fitting promoter. The shRNA is able to repress the expression of the GOI. However, a small guide RNA (sgRNA) with a sequence fitting the promoter upstream to the shRNA is also constitutively expressed. The sgRNA combines with the dCas9-KRAB to repress the expression of the shRNA, and in doing so represses its repression of the GOI.

In the fourth genetic circuit (FIG. 9D), the GOI is expressed under PPUT. A DNA-binding protein (GAL4) fused to the KRAB repressor (GAL4-KRAB) is expressed under the PPUT as well, be it on the same DNA fragment on a separate one. A small hairpin RNA (shRNA) with a sequence matching part of the RNA sequence from the RNA sequence of the GOI is constitutively expressed on a fitting promoter. The shRNA is able to repress the expression of the GOI. However, the transcription factor fused with the repressor (GAL4-KRAB) is able to attach to binding sites at the promoter upstream to the shRNA, and in doing so represses its repression of the GOI.

In the fifth genetic circuit (FIG. 9E), the GOI is expressed under PPUT. The GOI is co-expressed with a DNA-binding protein (GAL4) fused to the KRAB repressor (GAL4-KRAB), with an IRES in between the two genes. A shRNA with a sequence matching part of the RNA sequence from the RNA sequence of the GOI is constitutively expressed on a fitting promoter. The shRNA is able to repress the expression of the GOI. However, the transcription factor fused with the repressor (GAL4-KRAB) is able to attach to binding sites at the promoter upstream to the shRNA, and in doing so represses its repression of the GOI.

In the sixth genetic circuit (FIG. 9F), the GOI is expressed under PPUT. The GOI is co-expressed with a DNA-binding protein (GAL4) fused to the KRAB repressor (GAL4-KRAB), with an IRES between the two genes. A weak reverse promoter (in the figure shown with minimal CMV promoter) with binding sites for the transcription factor (GAL4) is placed in a reverse formation downstream to the gene. This placement has both a steric influence on the ability of the RNA polymerase working from the PPUT side to perform its transcription, as well as an indirect repression of the GOI by RNA binding of the reverse complementary sequence transcribed by the weak reverse promoter. However, the transcription factor fused with the repressor (GAL4-KRAB) is able to attach to binding sites at the weak reverse promoter, and in doing so represses its repression of the GOI.

In the seventh genetic circuit (FIG. 9G), the GOI is expressed under PPUT. The GOI is co-expressed with a transcription factor (TF), with an intron and a self-cleaving peptide sequence (shown as P2A) in between the two genes. The intron acts like a shRNA and has a sequence matching part of the RNA sequence from the RNA sequence of the GOI. The intron is able to repress the expression of the GOI and the TF. However, the TF is able to attach to binding sites at the promoter upstream to a BSS which in turn is able to repress the repression by the intron shRNA.

While certain features of the invention have been described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A system comprising at least 2 expression vectors, the system comprising:
a. a first expression vector comprising a first promoter sequence operably linked to a first nucleic acid sequence encoding a gene of interest (GOI) and a second nucleic acid sequence encoding a polynucleotide or a protein product thereof, characterized by being capable of inhibiting or reducing expression of a regulatory RNA polynucleotide, an activity thereof, or both, wherein said first promoter is responsive to an input signal; and
b. a second expression vector comprising a second promoter sequence operably linked to a nucleic acid sequence encoding said regulatory RNA polynucleotide, wherein said second promoter is constitutively active, and wherein said regulatory RNA polynucleotide inhibits or reduces expression levels of said GOI.

2. The system of claim 1, wherein said first expression vector further comprises a third nucleic acid sequence encoding a protein translation regulatory element.

3. The system of claim 2, wherein said third nucleic acid sequence is located between said first nucleic acid sequence and said second nucleic acid sequence.

4. The system of claim 1, further comprising a third expression vector comprising said first promoter sequence operably linked to a nucleic acid sequence encoding said polynucleotide or said protein product thereof, characterized by being capable of inhibiting or reducing expression of said regulatory RNA polynucleotide of said second expression vector, an activity thereof, or both.

5. The system of claim 4, wherein said first expression vector is devoid of said nucleic acid sequence encoding said polynucleotide or said protein product thereof, characterized by being capable of inhibiting or reducing expression of said regulatory RNA polynucleotide of said second expression vector, an activity thereof, or both.

6. A cell comprising the system of claim 1.

7. A method for controlling expression level of a GOI operably linked to an inducible promoter in a cell, wherein said promoter is responsive to an input signal, the method comprising contacting a cell with an effective amount of an agent triggering or providing said input signal, wherein said cell comprises a system comprising at least 2 expression vectors, the system comprising:
a. a first expression vector comprising a first promoter sequence operably linked to a first nucleic acid sequence encoding a gene of interest (GOI) and a second nucleic acid sequence encoding a polynucleotide or a protein product thereof, characterized by being capable of inhibiting or reducing expression of a regulatory RNA polynucleotide, an activity thereof, or both, wherein said first promoter is responsive to an input signal; and
b. a second expression vector comprising a second promoter sequence operably linked to a nucleic acid sequence encoding said regulatory RNA polynucleotide, wherein said second promoter is constitutively active, and wherein said regulatory RNA polynucleotide inhibits or reduces expression levels of said GOI,
thereby controlling expression level of the GOI operably linked to an inducible promoter in a cell.

8. The method of claim 7, wherein said controlling comprises reducing high basal expression level of said GOI in the absence of said agent, preserving high expression level of said GOI in the presence of said agent, or both.
